(19) United States
(12) Patent Application Publication
(10) Pub. No.: US 2025/0120981 A1
(43) Pub. Date: Apr. 17, 2025

(54) MRTX1133 PHARMACEUTICAL COMPOSITIONS

(71) Applicant: MIRATI THERAPEUTICS, INC., PRINCETON, NJ (US)

(72) Inventors: MICHALIS NICOLAOU, SAN DIEGO, CA (US); YOSYONG SURAKITBANBAM, SAN DIEGO, CA (US); SEAN PATRICK O'CONNELL, SAN DIEGO, CA (US)

(73) Assignee: MIRATI THERAPEUTICS, INC., PRINCETON, NJ (US)

(21) Appl. No.: 18/913,101

(22) Filed: Oct. 11, 2024

Related U.S. Application Data
(60) Provisional application No. 63/543,834, filed on Oct. 12, 2023.

(51) Int. Cl. A61K 31/55; A61K 9/48

(52) U.S. Cl. CPC A61K 31/55; A61K 9/4833; A61K 9/4858

(57) ABSTRACT

Solid pharmaceutical compositions of MRTX1133 (4-(4-((1R,5S)-3,8-diazabicyclo[3.2.1]octan-3-yl)-8-fluoro-2-(((2R,7aS)-2-fluorohexahydro-1Hpyrrolizin-7a-yl)methoxy)pyrido[4,3-d]pyrimidin-7-yl)-5-ethynyl-6-fluoronaphthalen-2-ol), including capsule and tablet formulations; methods for preparing these compositions, and methods of their use for the treatment of various diseases and disorders.

### CROSS REFERENCE TO RELATED APPLICATIONS

This application claims the benefit of U.S. Provisional Application No. 63/543,834, filed, Oct. 12, 2023, the entire content of which is hereby incorporated herein by reference.

### FIELD OF THE INVENTION

The present invention generally relates to solid pharmaceutical compositions of MRTX1133 (4-(4-((1R,5S)-3,8-diazabicyclo[3.2.1]octan-3-yl)-8-fluoro-2-(((2R,7aS)-2-fluorohexahydro-1Hpyrrolizin-7a-yl)methoxy)pyrido[4,3-d]pyrimidin-7-yl)-5-ethynyl-6-fluoronaphthalen-2-ol); methods for preparing these compositions, and methods of their use for the treatment of various diseases and disorders.

### BACKGROUND OF THE INVENTION

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors regulating a wide variety of processes, including cellular proliferation (e.g., see Alamgeer et al., (2013) Current Opin Pharmcol. 13:394-401).

The role of activated KRas in malignancy was observed over thirty years ago (e.g., see Santos et al., (1984) Science 223:661-664). Aberrant expression of KRas accounts for up to 20% of all cancers and oncogenic KRas mutations that stabilize GTP binding and lead to constitutive activation of KRas and downstream signaling have been reported in 25-30% of lung adenocarcinomas. (e.g., see Samatar and Poulikakos (2014) Nat Rev Drug Disc 13(12): 928-942 doi: 10.1038/nrd428). Single nucleotide substitutions that result in missense mutations at codons 12 and 13 of the KRas primary amino acid sequence comprise approximately 40% of these KRas driver mutations in lung adenocarcinoma. KRAS G12D mutation is present in 25.0% of all pancreatic ductal adenocarcinoma patients, 13.3% of all colorectal carcinoma patients, 10.1% of all rectal carcinoma patients, 4.1% of all non-small cell lung carcinoma patients and 1.7% of all small cell lung carcinoma patients (e.g., see The AACR Project GENIE Consortium, (2017) Cancer Discovery; 7(8):818-831. Dataset Version 4).

The well-known role of KRas in malignancy and the discovery of these frequent mutations in KRas in various tumor types made KRas a highly attractable target of the pharmaceutical industry for cancer therapy.

Compounds that inhibit KRas activity are still highly desirable and under investigation, including those that disrupt effectors such as guanine nucleotide exchange factors (e.g., see Sun et al., (2012) Agnew Chem Int Ed Engl. 51(25):6140-6143 doi: 10.1002/anie201201358) as well recent advances in the covalent targeting of an allosteric pocket of KRas G12C (e.g., see Ostrem et al., (2013) Nature 503:548-551 and Fell et al., (2018) ACS Med. Chem. Lett. 9:1230-1234). Clearly, there remains a continued interest and effort to develop inhibitors of KRas, particularly inhibitors of activating KRas mutants, especially KRas G12D.

A noncovalent inhibitor of KRas G12D is MRTX1133 (4-(4-((1R,5S)-3,8-diazabicyclo[3.2.1]octan-3-yl)-8-fluoro-2-(((2R,7aS)-2-fluorohexahydro-1Hpyrrolizin-7a-yl)methoxy)pyrido[4,3-d]pyrimidin-7-yl)-5-ethynyl-6-fluoronaphthalen-2-ol). An amorphous form of this compound was described in International Patent Application PCT/US2020/048194 filed Aug. 27, 2020 and published as WIPO publication WO2021/041671 on Mar. 4, 2021 at Example 252. The compound is also described in Qinheng Zheng et al, Identification of MRTX1133, a Noncovalent, Potent, and Selective KRAS^{G12D} Inhibitor, J. Med. Chem, 2022, 65, 4, 3123-3133.

A need therefore exists for a pharmaceutical composition of MRTX1133 which displays suitable bioavailability and shelf-life stability, which is not susceptible to liquid capsule leakage over time, and which has fewer side effects compared with capsule formulations.

### SUMMARY OF THE INVENTION

In one embodiment, the present invention provides a solid pharmaceutical composition, suitable for oral administration to a subject, including but not limited to a human subject, which comprises MRTX1133 or a pharmaceutically acceptable salt thereof, wherein the solid pharmaceutical composition, after administration to the subject, is capable of providing AUC_{0→∞} (the area under the curve of a plot of plasma drug concentration versus time) for MRTX1133 of at least, or about, 6900 ng*hr/mL.

In another embodiment, the solid pharmaceutical composition, after administration to a subject, is capable of providing AUC_{0→last} for MRTX1133 of at least, or about, 6700 ng*hr/mL.

In another embodiment, the solid pharmaceutical composition, after administration to a subject, is capable of providing AUC_{24→48} for MRTX1133 of at least, or about, 3900 ng*hr/mL.

In another embodiment, the solid pharmaceutical composition, after administration to a subject, is capable of providing AUC_{0→36} for MRTX1133 of at least, or about, 4700 ng*hr/mL.

In another embodiment, the solid pharmaceutical composition, after administration to a subject, is capable of providing AUC_{0→24} for MRTX1133 of at least, or about, 2800 ng*hr/mL.

In another embodiment, the solid pharmaceutical composition, after administration to a subject, is capable of providing AUC_{0→12} for MRTX1133 of at least, or about, 700 ng*hr/mL.

In another embodiment, the solid pharmaceutical composition, after administration to a subject, is capable of providing Cₘₐₓ for MRTX1133 of at least, or about, 710 ng/mL.

In another embodiment, the solid pharmaceutical composition, after administration to a subject, is capable of providing: a) AUC_{0→∞} for MRTX1133 of at least, or about, 6900 ng*hr/mL; b) AUC_{0→last} for MRTX1133 of at least, or about, 6700 ng*hr/mL; c) AUC_{24→48} for MRTX1133 of at least, or about, 3900 ng*hr/mL; d) AUC_{0→36} for MRTX1133 of at least, or about, 4700 ng*hr/mL; e) AUC_{0→24} for MRTX1133 of at least, or about, 2800 ng*hr/mL; f) AUC_{0→12} for MRTX1133 of at least, or about, 700 ng*hr/mL; and g) Cₘₐₓ for MRTX1133 of at least, or about, 710 ng/mL.

In another embodiment, the solid pharmaceutical composition provides a Tₘₐₓ of between about 14 hours and about 22 hours.

In any of the embodiments, it is not required that the recited pharmacokinetic (PK) values, such as AUC_{0→∞}, AUC_{0→last}, AUC_{24→48}, AUC_{0→36}, AUC_{0→24}, AUC_{0→12} and Cₘₐₓ, are achieved by administering a single pharmaceutical composition. The invention contemplates, and explicitly includes, embodiments where these PK values are achieved following administration of several capsule pharmaceutical compositions as a single dose (e.g., if a solid pharmaceutical composition comprises 150 mg MRTX1133, the single dose may include, for example, three of such pharmaceutical compositions, for the total administered amount of 450 mg MRTX1133).

In another embodiment, MRTX1133 is present as a salt thereof.

In another embodiment, the solid pharmaceutical composition comprises at least one additional anticancer compound in addition to MRTX1133.

In one embodiment, the solid pharmaceutical composition is in the form of a capsule.

In another embodiment, the solid pharmaceutical composition is in the form of a powder or a tablet, including an encapsulated powder.

In another embodiment, the solid pharmaceutical composition is in the form of a tablet.

In another embodiment, the tablet of the invention comprises a film coat.

In one embodiment, the pharmaceutical composition of the invention comprises MRTX1133 and sucrose acetate isobutyrate (“SAIB”).

In one embodiment, the capsule pharmaceutical composition of the invention comprises MRTX1133, SAIB, an antioxidant, a solvent, a surfactant, and an emulsifier.

In a preferred embodiment, an antioxidant comprises propyl gallate (PG).

In a preferred embodiment, the solvent comprises ethanol.

In a preferred embodiment, the surfactant is selected from the group consisting of polyoxylglycerides (e.g., caprylocaproyl Polyoxyl-8 glycerides), lauroyl polyoxyl-32 glycerides, and combinations thereof.

In a preferred embodiment, the emulsifier comprises D-α-tocopheryl polyethylene glycol succinate (Vitamin E TPGS).

In another embodiment, the tablet of the invention comprises: MRTX1133, one or more diluents, a disintegrant, a glidant, a lubricant, and a film coat.

In a preferred embodiment, the diluent is selected from the group consisting of microcrystalline cellulose, mannitol, and combinations thereof.

In another preferred embodiment, the disintegrant comprises crospovidone.

In another preferred embodiment, the glidant comprises colloidal silicon dioxide.

In another preferred embodiment, the lubricant comprises magnesium stearate.

In a preferred embodiment, the tablet of the invention comprises MRTX1133, microcrystalline cellulose, mannitol, crospovidone, colloidal silicon dioxide, magnesium stearate and a film coat.

In another embodiment, the solid pharmaceutical composition is provided as a unit dosage form.

In one embodiment, the amount of MRTX1133 in the solid pharmaceutical composition is at least, or about, 25 mg.

In one embodiment, the amount of MRTX1133 in the solid pharmaceutical composition is at least, or about, 50 mg.

In one embodiment, the amount of MRTX1133 in the solid pharmaceutical composition is at least, or about, 100 mg.

In one embodiment, the amount of MRTX1133 in the solid pharmaceutical composition is at least, or about, 150 mg.

In another embodiment, the amount of MRTX1133 in the solid pharmaceutical composition is at least, or about, 200 mg.

In another embodiment, the amount of MRTX1133 in the solid pharmaceutical composition is at least, or about, 300 mg.

In another embodiment, the amount of MRTX1133 in the solid pharmaceutical composition is at least, or about, 400 mg.

In another embodiment, the amount of MRTX1133 in the solid pharmaceutical composition is at least, or about, 600 mg.

In one embodiment, the tablets are extended release (ER) tablets.

In one embodiment, the amount of MRTX1133 in the composition is between 5-50%; preferably between 10-45%; more preferably between 15-40%, and most preferably between 20-40% by weight of the composition.

In another embodiment, the composition is in the form of a capsule and comprises:
(1) MRTX1133 constituting about 10-40% of the composition;
(2) SAIB constituting about 35-60% of the composition;
(3) an antioxidant constituting about 0-5% of the composition;
(4) a solvent constituting about 10% of the composition;
(5) an emulsifier constituting about 0-15% of the composition; and
(6) a surfactant constituting up to about 10-25% of the composition;
   wherein all percentages are percentages by weight and wherein the total weight is 100%.

In a preferred embodiment, MRTX1133 constitutes between about 10% and about 20% of the composition.

In another preferred embodiment, MRTX1133 constitutes about 15% of the composition.

In another preferred embodiment, MRTX1133 constitutes about 20% of the composition.

In another preferred embodiment, MRTX1133 constitutes about 35% of the composition.

In another embodiment, the composition is in the form of a capsule and comprises:
(1) MRTX1133 at about 20% of the composition;
(2) SAIB at about 35% of the composition;
(3) propyl gallate at about 5% of the composition;
(4) ethanol at about 10% of the composition;
(5) lauroyl polyoxyl-32 glycerides at about 5% of the composition;
(6) Vitamin E TPGS at about 10% of the composition; and
(7) Caprylocaproyl Polyoxyl-8 glycerides at about 15% of the composition;
   wherein all percentages are percentages by weight and wherein the total weight is 100%.

In another embodiment, the composition is in the form of a capsule and comprises:
(1) MRTX1133 at about 20% of the composition;
(2) SAIB at about 35% of the composition;
(3) propyl gallate at about 5% of the composition;
(4) ethanol at about 10% of the composition;
(5) lauroyl polyoxyl-32 glycerides at about 10% of the composition; and
(6) Vitamin E TPGS at about 20% of the composition;
   wherein all percentages are percentages by weight and wherein the total weight is 100%.

In another embodiment, the composition is in the form of a capsule and comprises:
(1) MRTX1133 at about 20% of the composition;
(2) SAIB at about 35% of the composition;
(3) propyl gallate at about 5% of the composition;
(4) ethanol at about 10% of the composition;
(5) Vitamin E TPGS at about 15% of the composition; and
(6) Caprylocaproyl Polyoxyl-8 glycerides at about 15% of the composition;
   wherein all percentages are percentages by weight and wherein the total weight is 100%.

In another embodiment, the composition is in the form of a capsule and comprises:
(1) MRTX1133 at about 20% of the composition;
(2) SAIB at about 35% of the composition;
(3) ethanol at about 10% of the composition;
(4) lauroyl polyoxyl-32 glycerides at about 5% of the composition;
(5) Vitamin E TPGS at about 15% of the composition; and
(6) Caprylocaproyl Polyoxyl-8 glycerides at about 15% of the composition;
   wherein all percentages are percentages by weight and wherein the total weight is 100%.

In another embodiment, the composition is in the form of a capsule and comprises:
(1) MRTX1133 at about 20% of the composition;
(2) SAIB at about 40% of the composition;
(3) propyl gallate at about 5% of the composition;
(4) ethanol at about 10% of the composition;
(5) lauroyl polyoxyl-32 glycerides at about 5% of the composition; and
(6) Caprylocaproyl Polyoxyl-8 glycerides at about 20% of the composition;
   wherein all percentages are percentages by weight and wherein the total weight is 100%.

In another embodiment, the composition is in the form of a tablet and comprises a film coat.

In another embodiment, the solid pharmaceutical composition is prepared by a process which comprises the steps of:
(a) mixing MRTX1133 and one or more solvents together to dissolve MRTX1133;
(b) adding an antioxidant, a surfactant and an emulsifier to the mixture of step (a) and stirring the mixture until all ingredients are dissolved;
(c) adding SAIB and a solvent to the mixture of step (b) and stirring until a uniform solution is formed;
(d) removing volatile solvents from the mixture of step (c); and
(e) encapsulating the solution of step (d) in capsules.

In one embodiment, the removal of volatile solvents in step (d) comprises reduced pressure evaporation.

In one embodiment, the solid pharmaceutical composition (e.g., a capsule or a tablet) may comprise additional excipients selected from the group consisting of diluents, fillers, super-disintegrants, binders, glidants, lubricants, and combinations thereof.

In another embodiment, the invention is directed to a method of treating cancer in a subject in need thereof, comprising orally administering to the subject a therapeutically effective amount of the solid pharmaceutical composition of the present invention. In one embodiment, the therapeutically effective amount is at least, or about, 25 mg of MRTX1133. In another embodiment, the therapeutically effective amount is at least, or about, 50 mg of MRTX1133. In another embodiment, the therapeutically effective amount is at least, or about, 100 mg of MRTX1133. In one embodiment, the therapeutically effective amount is at least, or about, 150 mg of MRTX1133. In one embodiment, the cancer is a KRas G12D-associated cancer. In one embodiment, the KRas G12D-associated cancer is lung cancer. In one embodiment, the solid pharmaceutical composition is a capsule.

Also provided herein are methods for treating cancer in a subject in need thereof, the method comprising (a) determining that cancer is associated with a KRas G12D mutation (e.g., a KRas G12D-associated cancer) (e.g., as determined using a regulatory agency-approved, e.g., FDA-approved, assay or kit); and (b) administering to the patient a therapeutically effective amount of the solid pharmaceutical composition of the present invention. In one embodiment, the solid pharmaceutical composition is a capsule.

### BRIEF DESCRIPTION OF THE DRAWINGS

FIG. 1 contains pharmacokinetic (PK) profiles of the tested formulations.

FIG. 2 is a graph of plasma concentration of MRTX1133 after MRTA191 dosing.

FIG. 3 is a graph of plasma concentration of MRTX1133 after MRTA193 dosing.

FIG. 4 is a graph of plasma concentration of MRTX1133 after MRTA194 dosing.

FIG. 5 is a graph of plasma concentration of MRTX1133 after MRTA195 dosing.

FIG. 6 is a graph of plasma concentration of MRTX1133 after MRTA196 dosing.

FIG. 7 is a graph of plasma concentration of MRTX1133 after MRTA197 dosing.

FIG. 8 is a graph of plasma concentration of MRTX1133 after MRTA132 dosing.

FIG. 9 is a graph of single-dose pharmacokinetic profiles of the evaluated doses of MRTX133 in humans.

FIG. 10 is a graph of single-dose pharmacokinetic profiles of the evaluated doses of MRTX133 in humans.

FIG. 11 shows single-dose and multiple dose pharmacokinetic profiles of the evaluated doses of MRTX133 in humans.

### DETAILED DESCRIPTION OF THE INVENTION

### Definitions

The term “MRTX1133” as used herein refers to a compound with the following formula:

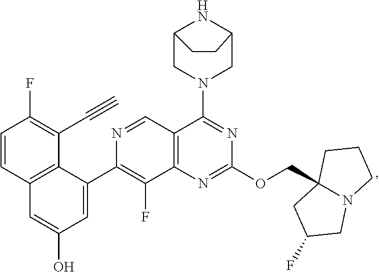 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

as well as pharmaceutically acceptable salts of this compound. The compound has the following chemical name: (4-(4-((1R,5S)-3,8-diazabicyclo[3.2.1]octan-3-yl)-8-fluoro-2-(((2R,7aS)-2-fluorohexahydro-1Hpyrrolizin-7a-yl)methoxy)pyrido[4,3-d]pyrimidin-7-yl)-5-ethynyl-6-fluoronaphthalen-2-ol). The invention encompasses amorphous and crystalline forms of MRTX1133. An amorphous form of this compound was described in International Patent Application PCT/US2020/048194 filed Aug. 27, 2020 and published as WIPO publication WO2021/041671 on Mar. 4, 2021 at Example 252. The compound is also described in Qinheng Zheng et al, Identification of MRTX1133, a Noncovalent, Potent, and Selective KRAS^{G12D} Inhibitor, J. Med. Chem, 2022, 65, 4, 3123-3133. The contents of this patent application and of the literature reference are hereby incorporated by reference in their entirety.

As used herein, “KRas G12D” refers to a mutant form of a mammalian KRas protein that contains an amino acid substitution of an aspartic acid for a glycine at amino acid position 12. The assignment of amino acid codon and residue positions for human KRas is based on the amino acid sequence identified by UniProtKB/Swiss-Prot P01116: Variant p.Glyl2Asp.

The term “composition” as used herein is intended to encompass a product comprising the specified ingredients (and in the specified amounts, if indicated), as well as any product which results, directly or indirectly, from combination of the specified ingredients in the specified amounts. By “pharmaceutically acceptable” it is meant the diluent, excipient or carrier must be compatible with the other ingredients of the formulation and not deleterious to the recipient thereof.

The term “solid composition” as used herein is intended to encompass capsules (including but not limited to gelatinous capsules containing viscous solution), tablets, powders, and other non-liquid formulations.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The term “crystalline” and related terms used herein, when used to describe a substance, component or product, means that the substance, component or product is crystalline as determined by X-ray diffraction. See, for example, Remington's Pharmaceutical Sciences, 18^{th} ed., Mack Publishing, Easton PA, p. 173 (1990); The United States Pharmacopeia, 23^{rd} ed., pp. 1843-1844 (1995); the contents of which are hereby incorporated by reference in their entireties.

The term “crystalline forms” and related terms herein refers to the various crystalline modifications of a given substance, including, but not limited to, polymorphs, solvates, hydrates, co-crystals and other molecular complexes, as well as salts, solvates of salts, hydrates of salts, other molecular complexes of salts, and polymorphs thereof.

The term “pharmaceutically acceptable salts” is meant to include salts of active compounds which are prepared with relatively nontoxic acids. Acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic; propionic; isobutyric; maleic; malonic; benzoic; succinic; suberic; fumaric; mandelic; phthalic; benzenesulfonic; toluenesulfonic, including p-toluenesulfonic, m-toluenesulfonic, and o-toluenesulfonic; citric; tartaric; methanesulfonic; and the like. Also included are salts of amino acids such as arginate and the like, and salts of organic acids like glucuronic or galactunoric acids and the like (see, for example, Berge et al. J. Pharm. Sci. 66:1-19 (1977)).

The term, “amorphous form,” as used herein, refers to a noncrystalline form of a substance.

The terms, “polymorphs” and “polymorphic forms” and related terms herein refer to crystal forms of a molecule. Different polymorphs may have different physical properties such as, for example, melting temperatures, heats of fusion, solubilities, dissolution rates and/or vibrational spectra as a result of the arrangement or conformation of the molecules in the crystal lattice. The differences in physical properties exhibited by polymorphs affect pharmaceutical parameters such as storage stability, compressibility and density (important in formulation and product manufacturing), and dissolution rates (an important factor in bioavailability). Polymorphs of a molecule can be obtained by a number of methods, as known in the art. Such methods include, but are not limited to, melt recrystallization, melt cooling, solvent recrystallization, desolvation, rapid evaporation, rapid cooling, slow cooling, vapor diffusion and sublimation.

Techniques for characterizing polymorphs include, but are not limited to, differential scanning calorimetry (DSC), X-ray powder diffractometry (XRPD), single crystal X-ray diffractometry, vibrational spectroscopy, e.g., IR and Raman spectroscopy, solid state NMR, hot stage optical microscopy, scanning electron microscopy (SEM), electron crystallography and quantitative analysis, particle size analysis (PSA), surface area analysis, solubility studies and dissolution studies.

The term, “solvate,” as used herein, refers to a crystal form of a substance which contains solvent. The term “hydrate” refers to a solvate wherein the solvent is water.

The term, “desolvated solvate,” as used herein, refers to a crystal form of a substance which can only be made by removing the solvent from a solvate.

The term “excipient” refers to an inactive ingredient of the pharmaceutical compositions of the invention. It includes, but is not limited to, solvents, wetting agents, diluents, superdisintegrants, binders, glidants, and lubricants.

The terms “treat”, “treating” or “treatment”, as used herein, refer to the reduction or amelioration of the progression, severity, and/or duration of a disorder or the eradication, reduction or amelioration of symptoms of a disorder, or the delay of the recurrence or onset of a disorder or one or more symptoms thereof in a subject that results from the administration of one or more compound.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having a KRas G12D mutation (e.g., as determined using a regulatory agency-approved, e.g., FDA-approved, assay or kit). In some embodiments, the subject has a tumor that is positive for a KRas G12D mutation (e.g., as determined using a regulatory agency-approved assay or kit). The subject can be a subject with a tumor(s) that is positive for a KRas G12D mutation (e.g., identified as positive using a regulatory agency-approved, e.g., FDA-approved, assay or kit). The subject can be a subject whose tumors have a KRas G12D mutation (e.g., where the tumor is identified as such using a regulatory agency-approved, e.g., FDA-approved, kit or assay). In some embodiments, the subject is suspected of having a KRas G12D gene-associated cancer. In some embodiments, the subject has a clinical record indicating that the subject has a tumor that has a KRas G12D mutation (and optionally the clinical record indicates that the subject should be treated with any of the compositions provided herein).

The term “pediatric patient” as used herein refers to a patient under the age of 16 years at the time of diagnosis or treatment. The term “pediatric” can be further divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994.

In some embodiments of any of the methods or uses described herein, an assay is used to determine whether the patient has KRas G12D mutation using a sample (e.g., a biological sample or a biopsy sample such as a paraffin-embedded biopsy sample) from a patient (e.g., a patient suspected of having a KRas G12D-associated cancer, a patient having one or more symptoms of a KRas G12D-associated cancer, and/or a patient that has an increased risk of developing a KRas G12D-associated cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR, quantitative real-time RT-PCR, allele-specific genotyping or ddPCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

As used herein, a “therapeutically effective amount” is an amount that is sufficient to ameliorate, or in some manner reduce a symptom or stop or reverse progression of a condition, or negatively modulate or inhibit the activity of KRas G12D. Such amount may be administered as a single dosage or may be administered according to a regimen, whereby it is effective.

As used herein, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The term, “AUC,” as used herein, refers to the area under the curve of a plot of plasma drug concentration versus time.

The term, “Tₘₐₓ,” as used herein, refers to the time after administration of a drug when the maximum plasma concentration is reached.

A “KRas G12D-associated disease or disorder” as used herein refers to diseases or disorders associated with or mediated by or having a KRas G12D mutation. A non-limiting example of a KRas G12D-associated disease or disorder is a KRas G12D-associated cancer.

### DETAILED DESCRIPTION OF COMPOSITIONS AND METHODS

The present invention is based on a surprising discovery that solid pharmaceutical compositions (e.g., capsules) comprising MRTX1133 and that also include SAIB (sucrose acetate isobutyrate) result in superior absorption of MRTX1133 as compared with pharmaceutical compositions that comprise MRTX1133 but do not comprise SAIB.

In one embodiment, the present invention provides a solid pharmaceutical composition, suitable for oral administration to a subject, including but not limited to a human subject, which comprises MRTX1133 or a pharmaceutically acceptable salt thereof, wherein the solid pharmaceutical composition, after administration to the subject, is capable of providing AUC_{0→∞} (the area under the curve of a plot of plasma drug concentration versus time) for MRTX1133 of at least, or about, 6900 ng*hr/mL.

In another embodiment, the solid pharmaceutical composition, after administration to a subject, is capable of providing AUC_{0→last} for MRTX1133 of at least, or about, 6700 ng*hr/mL.

In another embodiment, the solid pharmaceutical composition, after administration to a subject, is capable of providing AUC_{24→48} for MRTX1133 of at least, or about, 3900 ng*hr/mL.

In another embodiment, the solid pharmaceutical composition, after administration to a subject, is capable of providing AUC_{0→36} for MRTX1133 of at least, or about, 4700 ng*hr/mL.

In another embodiment, the solid pharmaceutical composition, after administration to a subject, is capable of providing AUC_{0→24} for MRTX1133 of at least, or about, 2800 ng*hr/mL.

In another embodiment, the solid pharmaceutical composition, after administration to a subject, is capable of providing AUC_{0→12} for MRTX1133 of at least, or about, 700 ng*hr/mL.

In another embodiment, the solid pharmaceutical composition, after administration to a subject, is capable of providing Cₘₐₓ for MRTX1133 of at least, or about, 710 ng/mL.

In another embodiment, the solid pharmaceutical composition, after administration to a subject, is capable of providing: a) AUC_{0→∞} for MRTX1133 of at least, or about, 6900 ng*hr/mL; b) AUC_{0→last} for MRTX1133 of at least, or about, 6700 ng*hr/mL; c) AUC_{24→48} for MRTX1133 of at least, or about, 3900 ng*hr/mL; d) AUC_{0→36} for MRTX1133 of at least, or about, 4700 ng*hr/mL; e) AUC_{0→24} for MRTX1133 of at least, or about, 2800 ng*hr/mL; f) AUC_{0→12} for MRTX1133 of at least, or about, 700 ng*hr/mL; and g) Cₘₐₓ for MRTX1133 of at least, or about, 710 ng/mL.

In another embodiment, the solid pharmaceutical composition provides a Tₘₐₓ of between about 14 hours and about 22 hours.

In any of the embodiments, it is not required that the recited pharmacokinetic (PK) values, such as AUC_{0→∞}, AUC_{0→last}, AUC_{24→48}, AUC_{0→36}, AUC_{0→24}, AUC_{0→12} and Cₘₐₓ, are achieved by administering a single pharmaceutical composition. The invention contemplates, and explicitly includes, embodiments where these PK values are achieved following administration of several capsule pharmaceutical compositions as a single dose (e.g., if a solid pharmaceutical composition comprises 150 mg MRTX1133, the single dose may include, for example, three of such pharmaceutical compositions, for the total administered amount of 450 mg MRTX1133).

In another embodiment, MRTX1133 is present as a salt thereof.

In another embodiment, the solid pharmaceutical composition comprises at least one additional anticancer compound in addition to MRTX1133.

In one embodiment, the solid pharmaceutical composition is in the form of a capsule.

In another embodiment, the solid pharmaceutical composition is in the form of a powder or a tablet, including an encapsulated powder.

In another embodiment, the solid pharmaceutical composition is in the form of a tablet.

In another embodiment, the tablet of the invention comprises a film coat.

In one embodiment, the pharmaceutical composition of the invention comprises MRTX1133 and sucrose acetate isobutyrate (“SAIB”).

In one embodiment, the capsule pharmaceutical composition of the invention comprises MRTX1133, SAIB, an antioxidant, a solvent, a surfactant, and an emulsifier.

In a preferred embodiment, an antioxidant comprises propyl gallate (PG).

In a preferred embodiment, the solvent comprises ethanol.

In a preferred embodiment, the surfactant is selected from the group consisting of polyoxylglycerides, lauroyl polyoxyl-32 glycerides, and combinations thereof.

In a preferred embodiment, the emulsifier comprises D-α-tocopheryl polyethylene glycol succinate (Vitamin E TPGS).

In another embodiment, the tablet of the invention comprises: MRTX1133, one or more diluents, a disintegrant, a glidant, a lubricant, and a film coat.

In a preferred embodiment, the diluent is selected from the group consisting of microcrystalline cellulose, mannitol, and combinations thereof.

In another preferred embodiment, the disintegrant comprises crospovidone.

In another preferred embodiment, the glidant comprises colloidal silicon dioxide.

In another preferred embodiment, the lubricant comprises magnesium stearate.

In a preferred embodiment, the tablet of the invention comprises MRTX1133, microcrystalline cellulose, mannitol, crospovidone, colloidal silicon dioxide, magnesium stearate and a film coat.

In another embodiment, the solid pharmaceutical composition is provided as a unit dosage form.

In one embodiment, the amount of MRTX1133 in the solid pharmaceutical composition is at least, or about, 25 mg.

In one embodiment, the amount of MRTX1133 in the solid pharmaceutical composition is at least, or about, 50 mg.

In one embodiment, the amount of MRTX1133 in the solid pharmaceutical composition is at least, or about, 100 mg.

In one embodiment, the amount of MRTX1133 in the solid pharmaceutical composition is at least, or about, 150 mg.

In another embodiment, the amount of MRTX1133 in the solid pharmaceutical composition is at least, or about, 200 mg.

In another embodiment, the amount of MRTX1133 in the solid pharmaceutical composition is at least, or about, 300 mg.

In another embodiment, the amount of MRTX1133 in the solid pharmaceutical composition is at least, or about, 400 mg.

In another embodiment, the amount of MRTX1133 in the solid pharmaceutical composition is at least, or about, 600 mg.

In one embodiment, the tablets are extended release (ER) tablets.

In one embodiment, the amount of MRTX1133 in the composition is between 5-50%; preferably between 10-45%; more preferably between 15-40%, and most preferably between 20-40% by weight of the composition.

In another embodiment, the composition is in the form of a capsule and comprises:
(1) MRTX1133 constituting about 10-40% of the composition;
(2) SAIB constituting about 35-60% of the composition;
(3) an antioxidant constituting about 0-5% of the composition;
(4) a solvent constituting about 10% of the composition;
(5) an emulsifier constituting about 0-15% of the composition; and
(6) a surfactant constituting up to about 10-25% of the composition;
   wherein all percentages are percentages by weight and wherein the total weight is 100%.

In a preferred embodiment, MRTX1133 constitutes between about 10% and about 20% of the composition.

In another preferred embodiment, MRTX1133 constitutes about 15% of the composition.

In another preferred embodiment, MRTX1133 constitutes about 20% of the composition.

In another preferred embodiment, MRTX1133 constitutes about 35% of the composition.

In another embodiment, the composition is in the form of a tablet and comprises a film coat.

In another embodiment, the solid pharmaceutical composition is prepared by a process which comprises the steps of:
(a) mixing MRTX1133 and one or more solvents together to dissolve MRTX1133;
(b) adding an antioxidant, a surfactant and an emulsifier to the mixture of step (a) and stirring the mixture until all ingredients are dissolved;
(c) adding SAIB and a solvent to the mixture of step (b) and stirring until a uniform solution is formed;
(d) removing volatile solvents from the mixture of step (c); and
(e) encapsulating the solution of step (d) in capsules.

In one embodiment, the removal of volatile solvents in step (d) comprises reduced pressure evaporation.

In one embodiment, the solid pharmaceutical composition (e.g., a capsule or a tablet) may comprise additional excipients selected from the group consisting of diluents, fillers, super-disintegrants, binders, glidants, lubricants, and combinations thereof.

Also provided herein are methods for treating cancer in a subject in need thereof, the method comprising (a) determining that cancer is associated with a KRas G12D mutation (e.g., a KRas G12D-associated cancer) (e.g., as determined using a regulatory agency-approved, e.g., FDA-approved, assay or kit); and (b) administering to the patient a therapeutically effective amount of the solid pharmaceutical composition of the present invention. In one embodiment, the solid pharmaceutical composition is a capsule.

It will be understood, however, that the specific dose level and frequency of dosage for any particular patient may be varied and will depend upon a variety of factors including, for example, the activity of the specific polymorph employed, the metabolic stability and length of action of that polymorph, the age, body weight, general health, sex, diet, mode and time of administration, rate of excretion, drug combination, and the severity of the patient's condition.

The pharmaceutical compositions provided herein can be combined with other compounds having related utilities to treat or prevent cancer. In many instances, administration of the subject pharmaceutical compositions in conjunction with these alternative agents enhances the efficacy of such agents. Accordingly, in some instances, the present pharmaceutical compositions, when combined or administered in combination with, e.g., anticancer agents, can be used in dosages which are less than the expected amounts when used alone, or less than the calculated amounts for combination therapy.

### EXAMPLES OF THE INVENTION

The following Examples illustrate the invention.

### Example 1: MRTX1133 Formulations

This example illustrates MRTX1133 solid pharmaceutical compositions.

### Materials and Methods

Undenatured, 200 proof, Ethyl Alcohol and Dichloromethane (DCM), HPLC grade, were purchased from Sigma Aldrich; Propyl Gallate NF was purchased from Spectrum Chemicals; Vitamin E TPGS was purchased from Antares Pharma; Gelucire® 44/14 (Lauroyl Polyoxyl-32 glycerides) and Labrasol® ALF (Caprylocaproyl Polyoxyl-8 glycerides) were provided by Gattefosse; Sucrose Acetate Isobutyrate (SAIB), BioSustane SAIB NF, was purchased from Eastman Chemicals; MRTX1133 (96%) was provided by Mirati Therapeutics Process Chemistry Group.

Instruments: Agilent 1260 HPLC equipped with a reverse phase column and method and Agilent Gas Chromatograph model. Reduced pressure evaporation equipment: Heidolph model Hei-VAP Core.

Table 1 illustrates the tested compositions.

TABLE 1
Drug Product, Unit Composition

| | % Content | | | | | | |
|---|---|---|---|---|---|---|---|
| Ingredient | MRTA132 | MRTA191 | MRTA193 | MRTA194 | MRTA195 | MRTA196 | MRTA197 |
| MRTX1133 | 10 | 20 | 20 | 20 | 20 | 20 | 20 |
| Propyl Gallate | 0 | 5 | 5 | 5 | 0 | 5 | 5 |
| Ethanol | 5 | 10 | 10 | 10 | 10 | 10 | 10 |
| Gelucire ® 44/14 | 5 | 5 | 10 | 0 | 5 | 10 | 5 |
| Vitamin E TPGS | 10 | 10 | 20 | 15 | 15 | 25 | 0 |
| Labrasol ® ALF | 15 | 15 | 0 | 15 | 15 | 30 | 20 |
| SAIB | 55 | 35 | 35 | 35 | 35 | 0 | 40 |
| Total | 100 | 100 | 100 | 100 | 100 | 100 | 100 |

### Preparation of MRTA132

5.50 grams of MRTX1133 were mixed by magnetic stirring with 62.2 g Ethanol to completely dissolve and form a dark reddish-brown solution. To this solution, Gelucire® 44/14, 2.47 g; Vitamin E TPGS, 5.06 g; and Labrasol® ALF, 7.53 g were added followed by stirring until all material dissolved and a reddish-brown solution was formed. To this solution, 29.5 g of previously prepared SAIB:Ethanol (9:1) solution, was added under stirring (29.5 g delivered 26.55 g of SAIB). The solution was allowed to stir for at least 15-30 mins or, until a uniform, slightly viscous solution was formed.

Removal of volatile solvent under reduced pressure evaporation afforded the following solvent level: Ethanol, 6.58%.

The resulting viscous solution was encapsulated in gelatin size 00 capsules containing approximately 700 mg formulation (targeted MRTX1133 strength: 70 mg).

MRTX1133 strength and purity were determined by HPLC; strength: 113.2 mg MRTX1133/g formulation and purity by HPLC was 99.61%.

### Preparation of MRTA191

27.08 grams of MRTX1133 were mixed by magnetic stirring with 81.2 g Ethanol and 54.00 g DCM to completely dissolve and form a dark reddish-brown solution. To this solution, propyl gallate, 6.43 g; Gelucire® 44/14, 6.43 g; Vitamin E TPGS, 12.89 g; and Labrasol® ALF, 19.28 g were added followed by stirring until all material dissolved and a reddish-brown solution was formed. To this solution, 52.32 g of previously prepared SAIB:Ethanol (9:1) solution, was added under stirring (52.32 g delivered 47.09 g of SAIB). The solution was allowed to stir for at least 15-30 mins or, until a uniform, slightly viscous solution was formed.

Removal of volatile solvents under reduced pressure evaporation afforded the following solvent levels: Ethanol, 10.19%; DCM, 412 ppm.

The resulting viscous solution was encapsulated in gelatin size 00 capsules containing approximately 750 mg formulation (targeted MRTX1133 strength: 150 mg).

MRTX1133 strength and purity were determined by HPLC; strength: 189.8 mg MRTX1133/g formulation and purity by HPLC was 97.85%.

### Preparation of MRTA193

9.57 grams of MRTX1133 were mixed by magnetic stirring with 26.82 g Ethanol and 17.84 g DCM to completely dissolve and form a dark reddish-brown solution. To this solution, propyl gallate, 2.23 g; Gelucire® 44/14, 2.28 g; Vitamin E TPGS, 10.15 g were added followed by stirring until all material dissolved and a reddish-brown solution was formed. To this solution, 19.85 g of previously prepared SAIB:Ethanol (9:1) solution, was added under stirring (19.84 g delivered 17.86 g of SAIB). The solution was allowed to stir for at least 15-30 mins or, until a uniform, slightly viscous solution was formed.

Removal of volatile solvents under reduced pressure evaporation afforded the following solvent levels: Ethanol, 9.23%; DCM, 281 ppm.

The resulting viscous solution was encapsulated in gelatin size 00 capsules containing approximately 750 mg formulation and 150 mg MRTX1133.

MRTX1133 strength and purity were determined by HPLC; strength: 190.7 mg MRTX1133/g MRTA193 formulation and purity by HPLC was 98.12%.

### Preparation of MRTA194

10.0 grams of MRTX1133 were mixed by magnetic stirring with 30.00 g Ethanol and 20.00 g DCM to completely dissolve and form a dark reddish-brown solution.

To this MRTX1133 solution, propyl gallate, 2.50 g; Vitamin E TPGS, 7.50 g; and Labrasol® ALF, 7.50 g were added followed by mixing under magnetic stirring until all solid material dissolved and a uniform dark reddish-brown solution was formed. To this solution 19.45 g, SAIB:Ethanol (9:1) solution was added under stirring (19.45 g delivered 17.50 g of SAIB). The solution was allowed to stir for at least 15-30 mins or, until a uniform, slightly viscous solution was formed.

Removal of volatile solvents under reduced pressure evaporation afforded the following solvent levels: Ethanol, 8.66%; DCM, 544 ppm.

The resulting viscous solution was encapsulated in gelatin size 00 capsules containing approximately 750 mg formulation and 150 mg MRTX1133.

MRTX1133 strength and purity were determined by HPLC; strength: 199 mg MRTX1133/g MRTA194 formulation and purity by HPLC was 98.54%.

### Preparation MRTA195

8.93 grams of MRTX1133 were mixed by magnetic stirring with 26.80 g Ethanol and 17.90 g DCM to completely dissolve and form a dark reddish-brown solution.

To this MRTX1133 solution, Gelucire® 44/14, 2.23 g; Vitamin E TPGS, 6.70 g; and Labrasol® ALF, 6.70 g were added followed by mixing under magnetic stirring until all solid material dissolved and a uniform dark reddish-brown solution was formed. To this solution 17.36 g, SAIB:Ethanol (9:1) solution was added under stirring (17.36 g delivered 15.63 g of SAIB). The solution was allowed to stir for at least 15-30 mins or, until a uniform, slightly viscous solution was formed.

Removal of volatile solvents under reduced pressure evaporation afforded the following solvent levels: Ethanol, 8.00%; DCM, 507 ppm.

The resulting viscous solution was encapsulated in gelatin size 00 capsules containing approximately 750 mg formulation and 150 mg MRTX1133.

MRTX1133 strength and purity were determined by HPLC; strength: 200.2 mg MRTX1133/g MRTA195 formulation and purity by HPLC was 98.35%.

### Preparation of MRTA196

8.93 grams of MRTX1133 was mixed by magnetic stirring with 28.48 g Ethanol and 19.02 g DCM to completely dissolve and form a dark reddish-brown solution.

To this MRTX1133 solution, propyl gallate, 2.22 g; Gelucire® 44/14, 4.50 g; Vitamin E TPGS, 11.18 g; and Labrasol® ALF, 13.50 g were added followed by mixing under magnetic stirring until all solid material dissolved and a uniform dark reddish-brown solution was formed. The solution was allowed to stir for at least 15-30 mins or, until a uniform, slightly viscous solution was formed.

Removal of volatile solvents under reduced pressure evaporation afforded the following solvent levels: DCM<600 ppm and ethanol 9-12% both determined by Gas Chromatography; (ethanol, 6.59%; DCM, 978 ppm).

Removal of volatile solvents under reduced pressure evaporation afforded the following solvent levels: Ethanol, 6.59%; DCM, 9.78 ppm.

The resulting viscous solution was encapsulated in gelatin size 00 capsules containing approximately 750 mg formulation (targeted MRTX1133 strength: 150 mg).

MRTX1133 strength and purity were determined by HPLC; strength (201.1 mg MRTX1133 per g MRTA196 formulation) and purity by HPLC was (98.46%).

### Preparation of MRTA197

9.00 grams of MRTX1133 were mixed by magnetic stirring with 27.01 g Ethanol and 18.03 g DCM to completely dissolve and form a dark reddish-brown solution.

To this MRTX1133 solution, propyl gallate, 2.28 g; Gelucire® 44/14, 2.25 g; and Labrasol® ALF, 9.01 g were added followed by mixing under magnetic stirring until all solid material dissolved and a uniform dark reddish-brown solution was formed.

To this solution, 20.01 g of previously prepared SAIB:Ethanol (9:1) solution, was added under stirring (20.01 g delivered 18.00 g of SAIB). The solution was allowed to stir for at least 15-30 mins or, until a uniform, slightly viscous solution was formed.

Removal of volatile solvents under reduced pressure evaporation afforded the following solvent levels: Ethanol, 10.02%; DCM, not detectable (ND).

The resulting viscous solution was encapsulated in gelatin size 00 capsules containing approximately 750 mg formulation and 150 mg MRTX1133.

MRTX1133 strength and purity were determined by HPLC; strength: 267.01 mg MRTX1133/g MRTA197 formulation and purity by HPLC was 98.08%.

### Example 2: Pharmacokinetics in Dogs

### Test System and Study Design

Three, non-naïve, fasted and pentagastrin-pretreated (IM injection, 6 μg/kg, 30 mins prior to each administration), male beagle dogs (≥8 kg and ≥6 months old) were administered 3 (three) 150 mg MRTX1133 (00 size) capsules, administered at T=0, 12, 24, 36 hours for a total of 6 capsules per day.

Blood Samples were Collected on:
T=0, 0.25, 0.5, 1, 2, 4, 8, 12, 13, 14, 15, 18, 24, 25, 26, 27, 30, 36, 37, 38, 39, 42 and 48 h.

Blood was collected into commercially available tubes (Jiangsu Kangjian Medical Supplies co., LTD) containing Potassium (K₂) EDTA (0.85-1.15 mg) gently inverted several times to ensure mixing and placed on wet ice until processed for plasma. Samples were centrifuged (3,200×g for 10 minutes at 2 to 8° C.) within 30 minutes from collection. The plasma sample (2*100 μL, one for analysis, one for backup) were transferred into labeled polypropylene micro-centrifuge tubes and stored frozen in a ≤−60° C. freezer until analyzed.

Test articles concentrations (ng/mL) in plasma were quantified by LC-MS/MS with internal standard.

### Results

Table 2 shows results of a bioavailability study of the tested compositions.

TABLE 2
Analysis of MRTX1133 Primary Pharmacokinetic Endpoints

| | MRTA191 | MRTA193 | MRTA194 | MRTA195 | MRTA196 | MRTA197 |
|---|---|---|---|---|---|---|
| Parameter | n = 3* | n = 3* | n = 3* | n = 3* | n = 3* | n = 3* |
| Cₘₐₓ (ng/mL) | 955 | 833 | 724 | 1012 | 388 | 638 |
| Tₘₐₓ (h) | 21.7 | 22.0 | 14.3 | 17.5 | 9.00 | 13.2 |
| T_{1/2} (h) | 2.81 | 3.51 | 3.00 | 4.07 | 8.54 | 4.17 |
| Tₗₐₛₜ (h) | 48.0 | 48.0 | 48.0 | 48.0 | 48.0 | 48.0 |
| AUC₀₋₁₂(h · ng/mL) | 1128 | 716 | 727 | 1033 | 567 | 1238 |
| AUC₀₋₂₄(h · ng/mL) | 3587 | 3198 | 2836 | 3119 | 1543 | 3829 |
| AUC₀₋₃₆(h · ng/mL) | 6908 | 6849 | 4777 | 5416 | 3042 | 6950 |
| AUC₂₄₋₄₈(h · ng/mL) | 5538 | 6030 | 3951 | 4720 | 2609 | 5384 |
| AUC₀₋ₗₐₛₜ (ng · h/mL) | 9125 | 9228 | 6787 | 7839 | 4152 | 9213 |
| AUC_{0-inf} (ng · h/mL) | 9320 | 9505 | 6988 | 8261 | 4888 | 9619 |
| MRT₀₋ₗₐₛₜ (h) | 25.3 | 26.1 | 25.3 | 25.3 | 25.7 | 25.0 |
| MRT_{0-inf} (h) | 25.8 | 26.9 | 26.0 | 26.8 | 32.6 | 26.1 |
| AUC_{Extra} (%) | 2.13 | 2.89 | 2.89 | 5.20 | 14.7 | 3.95 |
| AUMC_{Extra} (%) | 4.30 | 5.71 | 5.81 | 10.8 | 24.8 | 8.17 |

AUC₀₋₁₂ = area under the plasma concentration time curve from time 0 to 12 h;
AUC₀₋₂₄ = area under the plasma concentration time curve from time 0 to 24 h;
AUC₀₋₃₆ = area under the plasma concentration time curve from time 0 to 36 h;
AUC₂₄₋₄₈ = area under the plasma concentration time curve from time 24 to 48 h;
AUC₀₋ₗₐₛₜ = area under the plasma concentration time curve from time 0 to 48 h or the time of the last sample draw;
AUC_{0-inf} area under the plasma concentration time curve from time zero to infinity;
MRT₀₋ₗₐₛₜ mean residence time from 0 to the time of the last sample draw;
MRT_{0-inf} mean residence time from 0 to infinity;
AUC_{Extra} means a fraction of the total AUC that is due to the extrapolated AUC;
AUMC_{Extra} means a fraction of the total AUMC that is due to the extrapolated AUMC, wherein AUMC is the area under the moment curve

Pharmacokinetic studies in beagle dogs were designed to administer 450 mg dose twice daily for two days. From the results of the study, it is evident that the first 24 hours of administration, blood levels demonstrated the need for MRTX1133 equilibration; thus, only the second 24 hours are taken into consideration to assess success.

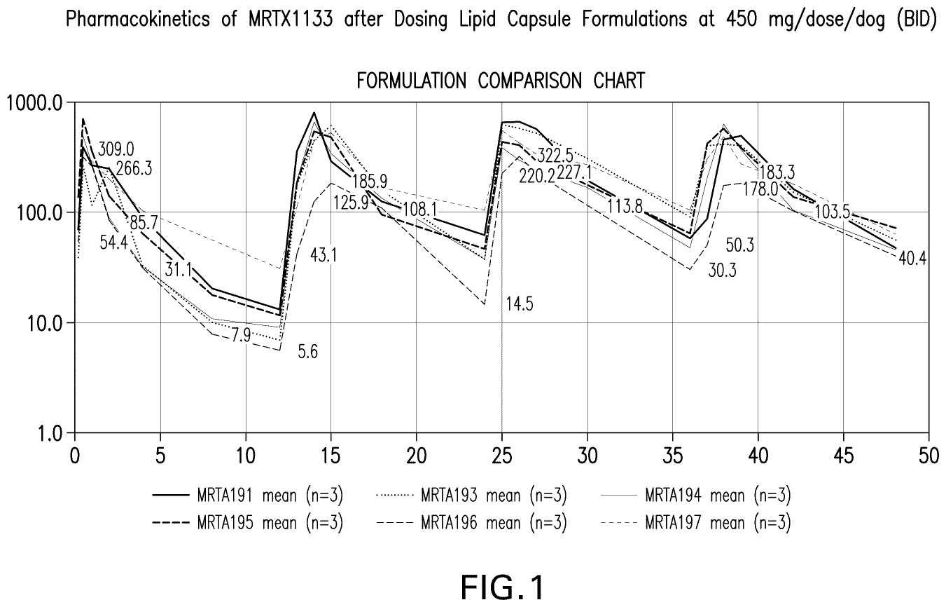

The pharmacokinetic profiles of the tested formulations are shown in FIG. 1. The X-axis is time (hours) and the Y-axis is blood concentration (ng/mL). The main measure of success of each formulation is the ability to deliver MRTX1133 in blood at levels above 100 ng/mL during the 12 hours post-administration time interval. Based on the data illustrated in FIG. 1, formulations MRTA191, MRTA193, MRTA194, MRTA195, MRTA197 show rather similar pharmacokinetic characteristics, delivering 100 ng/mL MRTX1133 for the majority of time post dosage administration. Formulation MRTA196, does not contain SAIB and absorption above the 100 ng/mL in Beagle dogs was not as desirable as formulations MRTA191, MRTA193, MRTA194, MRTA195 and MRTA197. Although for MRTA196 for short periods, 100 ng/mL blood concentration is reached, the majority of post administration time, blood concentration remains below the desirable level.

Based on the pharmacokinetic data presented in this study, it was surprisingly found that SAIB plays an important role in the absorption of MRTX1133 in comparison with all other ingredients used.

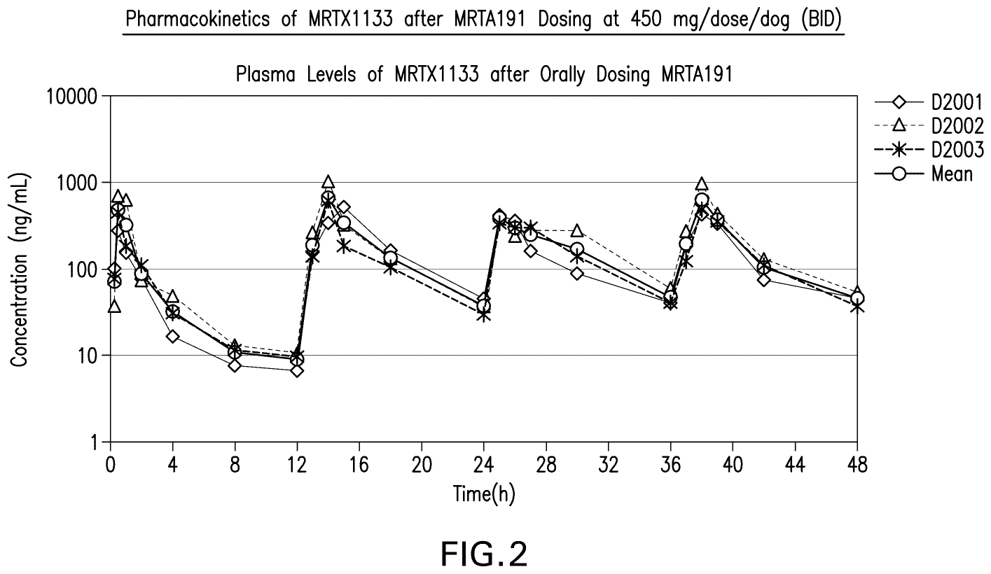

Table 3 lists pharmacokinetic data of MRTA191 formulation. FIG. 2 is a graph of plasma concentration of MRTX1133 after MRTA191 dosing.

TABLE 3
Summary of MRTA191 Pharmacokinetic Parameters
MRTA191

| | D1001 | D1002 | D1003 | Mean | | SD | CV (%) |
|---|---|---|---|---|---|---|---|
| Time (h) | | | | | | | |
| 0 | BQL | BQL | BQL | ND | ± | ND | ND |
| 0.25 | 87.4 | 272 | 135 | 165 | ± | 95.8 | 58.2 |
| 0.5 | 215 | 470 | 463 | 383 | ± | 145 | 38.0 |
| 1 | 527 | 90.6 | 181 | 266 | ± | 230 | 86.5 |
| 2 | 668 | 24.2 | 47.4 | 247 | ± | 365 | 148 |
| 4 | 212 | 17.0 | 36.8 | 88.6 | ± | 107 | 121 |
| 8 | 49.1 | 5.64 | 6.87 | 20.5 | ± | 24.7 | 120 |
| 12 | 30.9 | 3.82 | 4.59 | 13.1 | ± | 15.4 | 118 |
| 13 | 329 | 384 | 349 | 354 | ± | 27.8 | 7.86 |
| 14 | 1290 | 624 | 495 | 803 | ± | 427 | 53.1 |
| 15 | 464 | 210 | 192 | 289 | ± | 152 | 52.7 |
| 18 | 267 | 59.0 | 48.0 | 125 | ± | 123 | 99.0 |
| 24 | 86.0 | 38.4 | 62.2 | 62.2 | ± | 23.8 | 38.3 |
| 25 | 1440 | 270 | 250 | 653 | ± | 681 | 104 |
| 26 | 673 | 520 | 801 | 665 | ± | 141 | 21.2 |
| 27 | 438 | 567 | 700 | 568 | ± | 131 | 23.1 |
| 30 | 198 | 176 | 212 | 195 | ± | 18.1 | 9.29 |
| 36 | 72.1 | 52.7 | 49.7 | 58.2 | ± | 12.2 | 20.9 |
| 37 | 91.3 | 81.0 | 88.5 | 86.9 | ± | 5.33 | 6.13 |
| 38 | 487 | 523 | 366 | 459 | ± | 82.2 | 17.9 |
| 39 | 579 | 386 | 511 | 492 | ± | 97.9 | 19.9 |
| 42 | 216 | 181 | 95.4 | 164 | ± | 62.0 | 37.8 |
| 48 | 63.6 | 42.8 | 37.0 | 47.8 | ± | 14.0 | 29.3 |
| PK Parameters | | | | | | | |
| Rsq_adj | 0.967 | 1.00 | 0.848 | — | ± | — | — |
| No. points used for T_{1/2} | 3.00 | 3.00 | 4.00 | ND | ± | — | — |
| Cₘₐₓ (ng/mL) | 1440 | 624 | 801 | 955 | ± | 429 | 44.9 |
| Tₘₐₓ (h) | 25.0 | 14.0 | 26.0 | 21.7 | ± | 6.66 | 30.7 |
| T_{1/2} (h) | 2.89 | 2.84 | 2.70 | 2.81 | ± | 0.0985 | 3.50 |
| Tₗₐₛₜ (h) | 48.0 | 48.0 | 48.0 | 48.0 | ± | — | — |
| AUC₀₋₁₂(h · ng/mL) | 2394 | 430 | 561 | 1128 | ± | 1098 | 97.3 |
| AUC₀₋₂₄(h · ng/mL) | 6416 | 2152 | 2194 | 3587 | ± | 2450 | 68.3 |
| AUC₀₋₃₆(h · ng/mL) | 10389 | 4812 | 5522 | 6908 | ± | 3036 | 43.9 |
| AUC₂₄₋₄₈(h · ng/mL) | 6675 | 4800 | 5138 | 5538 | ± | 999 | 18.0 |
| AUC₀₋ₗₐₛₜ (ng · h/mL) | 13091 | 6953 | 7332 | 9125 | ± | 3440 | 37.7 |
| AUC_{0-inf} (ng · h/mL) | 13356 | 7128 | 7476 | 9320 | ± | 3500 | 37.5 |
| MRT₀₋ₗₐₛₜ (h) | 22.2 | 27.3 | 26.3 | 25.3 | ± | 2.70 | 10.7 |
| MRT_{0-inf} (h) | 22.8 | 27.9 | 26.8 | 25.8 | ± | 2.68 | 10.4 |
| AUC_{Extra} (%) | 1.99 | 2.46 | 1.93 | 2.13 | ± | 0.290 | 13.6 |
| AUMC_{Extra} (%) | 4.55 | 4.61 | 3.73 | 4.30 | ± | 0.492 | 11.4 |

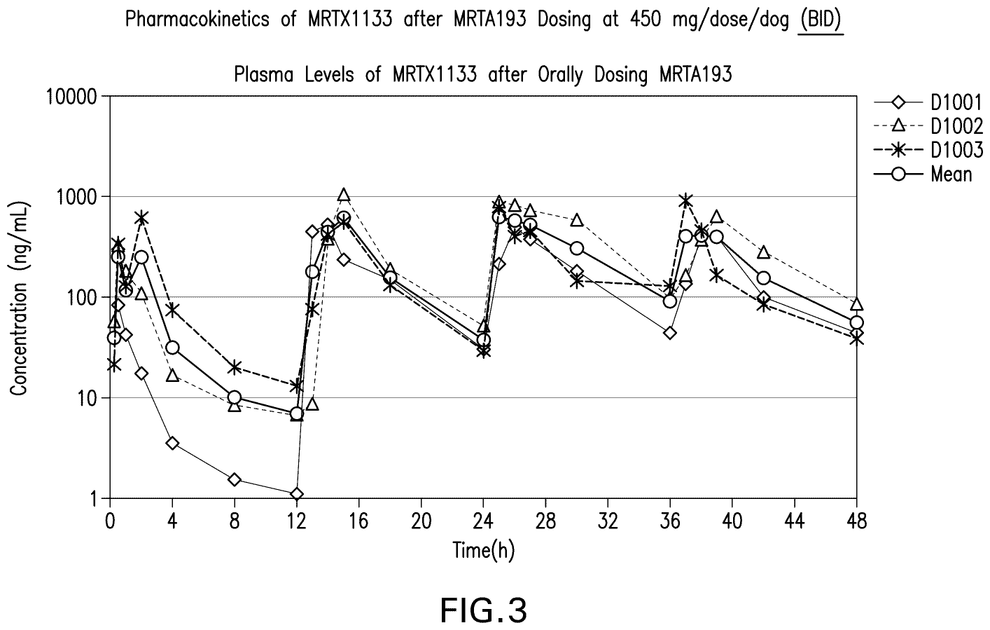

Table 4 lists pharmacokinetic data of MRTA193 formulation. FIG. 3 is a graph of plasma concentration of MRTX1133 after MRTA193 dosing.

TABLE 4
Summary of MRTA193 Pharmacokinetic Parameters
MRTA193

| | D1001 | D1002 | D1003 | Mean | | SD | CV (%) |
|---|---|---|---|---|---|---|---|
| Time (h) | | | | | | | |
| 0 | BQL | BQL | BQL | ND | ± | ND | ND |
| 0.25 | BQL | 57.7 | 21.5 | 39.6 | ± | ND | ND |
| 0.5 | 83.6 | 327 | 341 | 251 | ± | 145 | 57.8 |
| 1 | 42.5 | 183 | 126 | 117 | ± | 70.7 | 60.3 |
| 2 | 17.5 | 109 | 623 | 250 | ± | 326 | 131 |
| 4 | 3.56 | 16.8 | 74.9 | 31.8 | ± | 37.9 | 120 |
| 8 | 1.56 | 8.48 | 20.1 | 10.0 | ± | 9.37 | 93.3 |
| 12 | 1.11 | 6.82 | 13.0 | 6.98 | ± | 5.95 | 85.2 |
| 13 | 453 | 8.75 | 76.5 | 179 | ± | 239 | 133 |
| 14 | 529 | 381 | 424 | 445 | ± | 76.1 | 17.1 |
| 15 | 234 | 1060 | 542 | 612 | ± | 417 | 68.2 |
| 18 | 147 | 190 | 133 | 157 | ± | 29.7 | 19.0 |
| 24 | 30.7 | 52.2 | 29.5 | 37.5 | ± | 12.8 | 34.1 |
| 25 | 213 | 887 | 777 | 626 | ± | 362 | 57.8 |
| 26 | 495 | 825 | 409 | 576 | ± | 220 | 38.1 |
| 27 | 377 | 733 | 451 | 520 | ± | 188 | 36.1 |
| 30 | 182 | 590 | 147 | 306 | ± | 246 | 80.4 |
| 36 | 44.3 | 97.9 | 130 | 90.7 | ± | 43.3 | 47.7 |
| 37 | 136 | 165 | 909 | 403 | ± | 438 | 109 |
| 38 | 411 | 372 | 455 | 413 | ± | 41.5 | 10.1 |
| 39 | 397 | 635 | 168 | 400 | ± | 234 | 58.4 |
| 42 | 100 | 280 | 85.3 | 155 | ± | 108 | 69.9 |
| 48 | 44.1 | 86.5 | 38.8 | 56.5 | ± | 26.1 | 46.3 |
| PK Parameters | | | | | | | |
| Rsq_adj | 0.886 | 0.985 | 0.957 | — | ± | — | — |
| No. points used for T_{1/2} | 4.00 | 3.00 | 3.00 | ND | ± | — | — |
| Cₘₐₓ (ng/mL) | 529 | 1060 | 909 | 833 | ± | 274 | 32.9 |
| Tₘₐₓ (h) | 14.0 | 15.0 | 37.0 | 22.0 | ± | 13.0 | 59.1 |
| T_{1/2} (h) | 2.96 | 3.18 | 4.38 | 3.51 | ± | 0.764 | 21.8 |
| Tₗₐₛₜ (h) | 48.0 | 48.0 | 48.0 | 48.0 | ± | — | — |
| AUC₀₋₁₂ (ng · h/mL) | 119 | 536 | 1493 | 716 | ± | 705 | 98.4 |
| AUC₀₋₂₄ (ng · h/mL) | 2206 | 3617 | 3771 | 3198 | ± | 863 | 27.0 |
| AUC₀₋₃₆ (ng · h/mL) | 4456 | 9166 | 6926 | 6849 | ± | 2356 | 34.4 |
| AUC₂₄₋₄₈ (ng · h/mL) | 4040 | 8713 | 5338 | 6030 | ± | 2412 | 40.0 |
| AUC₀₋ₗₐₛₜ (ng · h/mL) | 6246 | 12330 | 9109 | 9228 | ± | 3044 | 33.0 |
| AUC_{0-inf} (ng · h/mL) | 6434 | 12727 | 9354 | 9505 | ± | 3149 | 33.1 |
| MRT₀₋ₗₐₛₜ (h) | 27.1 | 27.6 | 23.5 | 26.1 | ± | 2.24 | 8.58 |
| MRT_{0-inf} (h) | 27.9 | 28.4 | 24.3 | 26.9 | ± | 2.24 | 8.33 |
| AUC_{Extra} (%) | 2.93 | 3.12 | 2.62 | 2.89 | ± | 0.252 | 8.73 |
| AUMC_{Extra} (%) | 5.50 | 5.78 | 5.85 | 5.71 | ± | 0.185 | 3.24 |

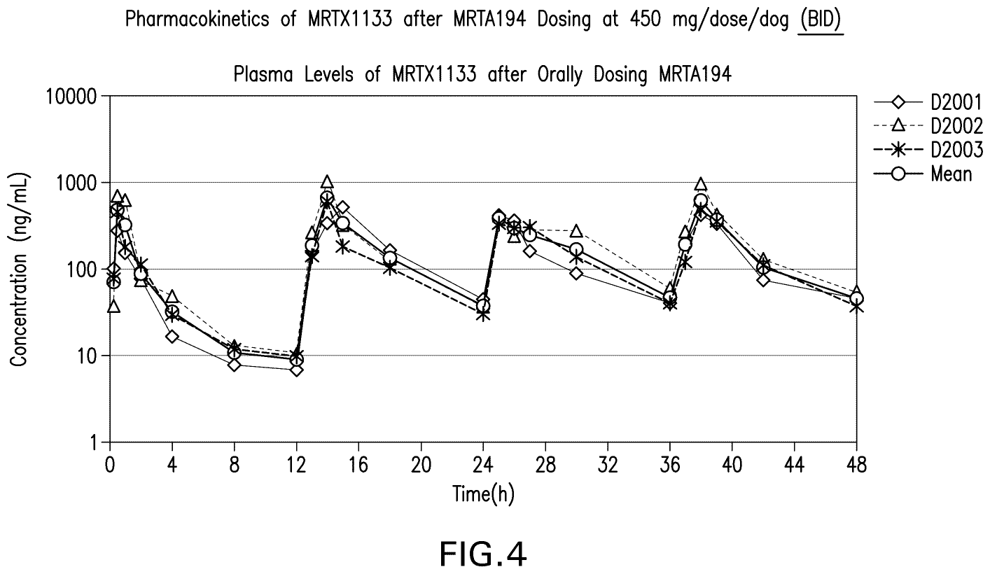

Table 5 lists pharmacokinetic data of MRTA194 formulation. FIG. 4 is a graph of plasma concentration of MRTX1133 after MRTA194 dosing.

TABLE 5
Summary of MRTA194 Pharmacokinetic Parameters
MRTA194

| | D2001 | D2002 | D2003 | Mean | | SD | CV (%) |
|---|---|---|---|---|---|---|---|
| Time (h) | | | | | | | |
| 0 | BQL | BQL | BQL | ND | ± | ND | ND |
| 0.25 | 101 | 37.8 | 75.8 | 71.5 | ± | 31.8 | 44.5 |
| 0.5 | 278 | 703 | 458 | 480 | ± | 213 | 44.5 |
| 1 | 153 | 628 | 184 | 322 | ± | 266 | 82.6 |
| 2 | 77.4 | 73.9 | 112 | 87.8 | ± | 21.1 | 24.0 |
| 4 | 16.6 | 48.8 | 29.9 | 31.8 | ± | 16.2 | 50.9 |
| 8 | 7.77 | 12.9 | 11.8 | 10.8 | ± | 2.70 | 25.0 |
| 12 | 6.77 | 10.6 | 9.72 | 9.03 | ± | 2.01 | 22.2 |
| 13 | 157 | 266 | 141 | 188 | ± | 68.0 | 36.2 |
| 14 | 338 | 1030 | 622 | 663 | ± | 348 | 52.4 |
| 15 | 519 | 325 | 183 | 342 | ± | 169 | 49.3 |
| 18 | 165 | 133 | 104 | 134 | ± | 30.5 | 22.8 |
| 24 | 45.0 | 37.1 | 30.6 | 37.6 | ± | 7.21 | 19.2 |
| 25 | 415 | 414 | 340 | 390 | ± | 43.0 | 11.0 |
| 26 | 358 | 242 | 301 | 300 | ± | 58.0 | 19.3 |
| 27 | 161 | 280 | 302 | 248 | ± | 75.9 | 30.6 |
| 30 | 88.6 | 276 | 141 | 169 | ± | 96.7 | 57.4 |
| 36 | 40.7 | 59.6 | 41.4 | 47.2 | ± | 10.7 | 22.7 |
| 37 | 185 | 275 | 121 | 194 | ± | 77.4 | 39.9 |
| 38 | 425 | 970 | 486 | 627 | ± | 299 | 47.6 |
| 39 | 333 | 429 | 352 | 371 | ± | 50.8 | 13.7 |
| 42 | 74.4 | 128 | 110 | 104 | ± | 27.3 | 26.2 |
| 48 | 46.9 | 53.6 | 37.4 | 46.0 | ± | 8.14 | 17.7 |
| PK Parameters | | | | | | | |
| Rsq_adj | 0.770 | 0.843 | 0.946 | — | ± | — | — |
| No. points used for T_{1/2} | 4.00 | 3.00 | 4.00 | ND | ± | — | — |
| Cₘₐₓ (ng/mL) | 519 | 1030 | 622 | 724 | ± | 270 | 37.3 |
| Tₘₐₓ (h) | 15.0 | 14.0 | 14.0 | 14.3 | ± | 0.577 | 4.03 |
| T_{1/2} (h) | 3.11 | 3.17 | 2.71 | 3.00 | ± | 0.250 | 8.34 |
| Tₗₐₛₜ (h) | 48.0 | 48.0 | 48.0 | 48.0 | ± | — | — |
| AUC₀₋₁₂(h · ng/mL) | 455 | 1074 | 653 | 727 | ± | 316 | 43.5 |
| AUC₀₋₂₄(h · ng/mL) | 2694 | 3567 | 2248 | 2836 | ± | 671 | 23.7 |
| AUC₀₋₃₆(h · ng/mL) | 4226 | 5985 | 4120 | 4777 | ± | 1048 | 21.9 |
| AUC₂₄₋₄₈(h · ng/mL) | 3168 | 5033 | 3652 | 3951 | ± | 968 | 24.5 |
| AUC₀₋ₗₐₛₜ (ng · h/mL) | 5862 | 8600 | 5900 | 6787 | ± | 1570 | 23.1 |
| AUC_{0-inf} (ng · h/mL) | 6072 | 8845 | 6047 | 6988 | ± | 1608 | 23.0 |
| MRT₀₋ₗₐₛₜ (h) | 25.0 | 25.1 | 25.7 | 25.3 | ± | 0.379 | 1.50 |
| MRT_{0-inf} (h) | 25.9 | 25.8 | 26.4 | 26.0 | ± | 0.321 | 1.23 |
| AUC_{Extra} (%) | 3.47 | 2.77 | 2.42 | 2.89 | ± | 0.535 | 18.5 |
| AUMC_{Extra} (%) | 7.01 | 5.64 | 4.77 | 5.81 | ± | 1.13 | 19.4 |

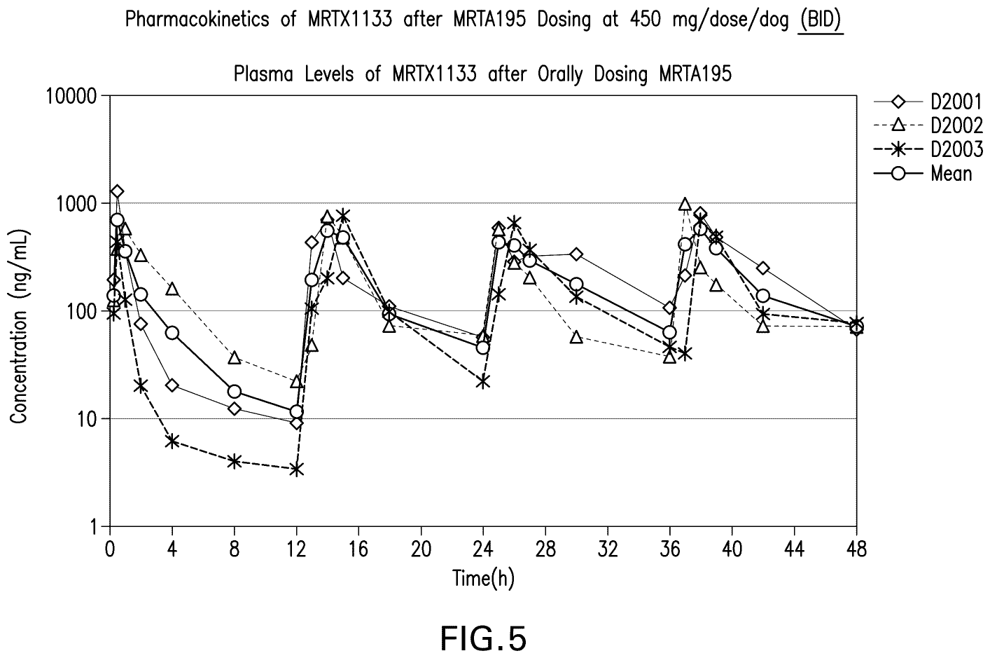

Table 6 lists pharmacokinetic data of MRTA195 formulation. FIG. 5 is a graph of plasma concentration of MRTX1133 after MRTA195 dosing.

TABLE 6
Summary of MRTA195 Pharmacokinetic Parameters
MRTA195

| | D2001 | D2002 | D2003 | Mean | | SD | CV (%) |
|---|---|---|---|---|---|---|---|
| Time (h) | | | | | | | |
| 0 | BQL | BQL | BQL | ND | ± | ND | ND |
| 0.25 | 196 | 126 | 94.9 | 139 | ± | 51.8 | 37.3 |
| 0.5 | 1290 | 379 | 435 | 701 | ± | 511 | 72.8 |
| 1 | 371 | 580 | 127 | 359 | ± | 227 | 63.1 |
| 2 | 76.5 | 333 | 20.2 | 143 | ± | 167 | 116 |
| 4 | 20.4 | 162 | 6.19 | 62.9 | ± | 86.1 | 137 |
| 8 | 12.4 | 37.2 | 4.03 | 17.9 | ± | 17.2 | 96.5 |
| 12 | 9.18 | 22.2 | 3.41 | 11.6 | ± | 9.63 | 83.0 |
| 13 | 430 | 48.4 | 105 | 194 | ± | 206 | 106 |
| 14 | 698 | 764 | 202 | 555 | ± | 307 | 55.4 |
| 15 | 201 | 482 | 757 | 480 | ± | 278 | 57.9 |
| 18 | 110 | 73.1 | 98.9 | 94.0 | ± | 18.9 | 20.1 |
| 24 | 57.5 | 58.3 | 22.3 | 46.0 | ± | 20.6 | 44.7 |
| 25 | 593 | 575 | 143 | 437 | ± | 255 | 58.3 |
| 26 | 287 | 282 | 653 | 407 | ± | 213 | 52.2 |
| 27 | 309 | 204 | 369 | 294 | ± | 83.5 | 28.4 |
| 30 | 339 | 57.5 | 136 | 178 | ± | 145 | 81.8 |
| 36 | 107 | 37.6 | 46.5 | 63.7 | ± | 37.8 | 59.3 |
| 37 | 215 | 989 | 40.9 | 415 | ± | 505 | 122 |
| 38 | 806 | 253 | 682 | 580 | ± | 290 | 50.0 |
| 39 | 484 | 175 | 490 | 383 | ± | 180 | 47.0 |
| 42 | 250 | 72.4 | 94.9 | 139 | ± | 96.7 | 69.5 |
| 48 | 67.7 | 71.5 | 77.0 | 72.1 | ± | 4.68 | 6.49 |
| PK Parameters | | | | | | | |
| Rsq_adj | 1.00 | 0.549 | 0.656 | — | ± | — | — |
| No. points used for T_{1/2} | 3.00 | 4.00 | 4.00 | ND | ± | — | — |
| Cₘₐₓ (ng/mL) | 1290 | 989 | 757 | 1012 | ± | 267 | 26.4 |
| Tₘₐₓ (h) | 0.500 | 37.0 | 15.0 | 17.5 | ± | 18.4 | 105 |
| T_{1/2} (h) | 3.17 | 5.85 | 3.19 | 4.07 | ± | 1.54 | 37.9 |
| Tₗₐₛₜ (h) | 48.0 | 48.0 | 48.0 | 48.0 | ± | — | — |
| AUC₀₋₁₂ (ng · h/mL) | 958 | 1787 | 354 | 1033 | ± | 720 | 69.7 |
| AUC₀₋₂₄ (ng · h/mL) | 2958 | 4079 | 2320 | 3119 | ± | 890 | 28.6 |
| AUC₀₋₃₆ (ng · h/mL) | 6085 | 5744 | 4419 | 5416 | ± | 880 | 16.3 |
| AUC₂₄₋₄₈ (ng · h/mL) | 6261 | 3711 | 4187 | 4720 | ± | 1356 | 28.7 |
| AUC₀₋ₗₐₛₜ (ng · h/mL) | 9219 | 7790 | 6507 | 7839 | ± | 1357 | 17.3 |
| AUC_{0-inf} (ng · h/mL) | 9528 | 8393 | 6861 | 8261 | ± | 1338 | 16.2 |
| MRT₀₋ₗₐₛₜ (h) | 27.3 | 21.5 | 27.1 | 25.3 | ± | 3.29 | 13.0 |
| MRT_{0-inf} (h) | 28.1 | 24.0 | 28.4 | 26.8 | ± | 2.46 | 9.16 |
| AUC_{Extra} (%) | 3.25 | 7.19 | 5.16 | 5.20 | ± | 1.97 | 37.9 |
| AUMC_{Extra} (%) | 6.08 | 16.9 | 9.56 | 10.8 | ± | 5.52 | 50.9 |

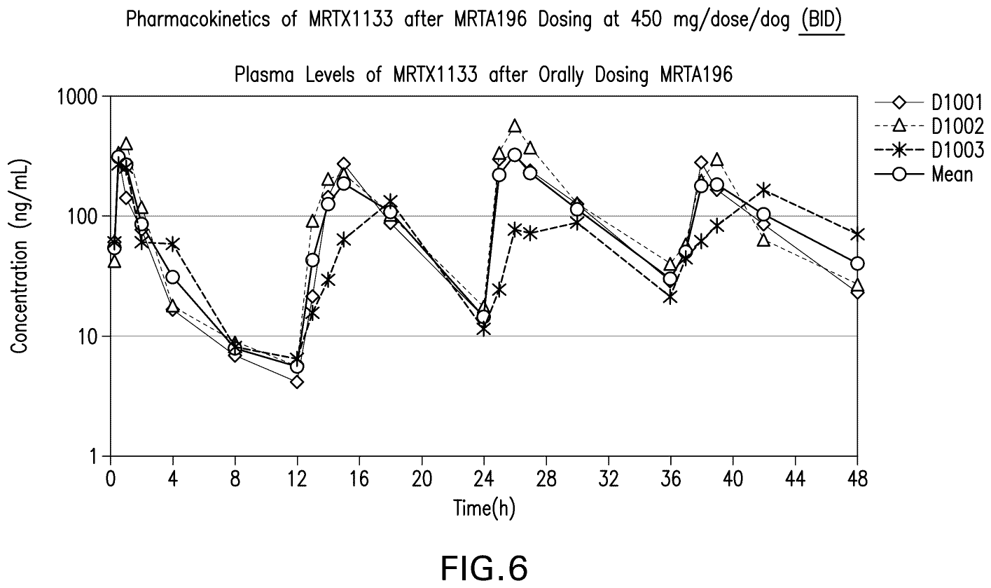

Table 7 lists pharmacokinetic data of MRTA196 formulation. FIG. 6 is a graph of plasma concentration of MRTX1133 after MRTA196 dosing.

TABLE 7
Summary of MRTA196 Pharmacokinetic Parameters
MRTA196

| | D1001 | D1002 | D1003 | Mean | | SD | CV (%) |
|---|---|---|---|---|---|---|---|
| Time (h) | | | | | | | |
| 0 | BQL | BQL | BQL | ND | ± | ND | ND |
| 0.25 | 60.6 | 42.5 | 60.1 | 54.4 | ± | 10.3 | 18.9 |
| 0.5 | 320 | 334 | 273 | 309 | ± | 32.0 | 10.3 |
| 1 | 141 | 402 | 256 | 266 | ± | 131 | 49.1 |
| 2 | 77.8 | 119 | 60.4 | 85.7 | ± | 30.1 | 35.1 |
| 4 | 16.8 | 18.1 | 58.3 | 31.1 | ± | 23.6 | 75.9 |
| 8 | 6.94 | 8.84 | 8.04 | 7.94 | ± | 0.954 | 12.0 |
| 12 | 4.18 | 6.17 | 6.41 | 5.59 | ± | 1.22 | 21.9 |
| 13 | 21.6 | 91.9 | 15.8 | 43.1 | ± | 42.4 | 98.3 |
| 14 | 143 | 205 | 29.6 | 126 | ± | 88.9 | 70.7 |
| 15 | 273 | 221 | 63.7 | 186 | ± | 109 | 58.6 |
| 18 | 88.3 | 104 | 132 | 108 | ± | 22.1 | 20.5 |
| 24 | 14.5 | 17.5 | 11.6 | 14.5 | ± | 2.95 | 20.3 |
| 25 | 299 | 337 | 24.5 | 220 | ± | 171 | 77.4 |
| 26 | 320 | 570 | 77.5 | 323 | ± | 246 | 76.4 |
| 27 | 238 | 371 | 72.4 | 227 | ± | 150 | 65.9 |
| 30 | 126 | 127 | 88.4 | 114 | ± | 22.0 | 19.3 |
| 36 | 29.3 | 40.2 | 21.3 | 30.3 | ± | 9.49 | 31.3 |
| 37 | 48.2 | 58.2 | 44.6 | 50.3 | ± | 7.05 | 14.0 |
| 38 | 278 | 194 | 62.1 | 178 | ± | 109 | 61.1 |
| 39 | 166 | 300 | 83.9 | 183 | ± | 109 | 59.5 |
| 42 | 85.7 | 63.9 | 161 | 104 | ± | 50.9 | 49.2 |
| 48 | 23.2 | 27.1 | 70.8 | 40.4 | ± | 26.4 | 65.5 |
| PK Parameters | | | | | | | |
| Rsq_adj | 1.00 | 0.820 | −0.715 | — | ± | — | — |
| No. points used for T_{1/2} | 3.00 | 4.00 | 3.00 | ND | ± | — | — |
| Cₘₐₓ (ng/mL) | 320 | 570 | 273 | 388 | ± | 160 | 41.2 |
| Tₘₐₓ (h) | 0.500 | 26.0 | 0.500 | 9.00 | ± | 14.7 | 164 |
| T_{1/2} (h) | 3.17 | 3.06 | 19.4 | 8.54 | ± | 9.40 | 110 |
| Tₗₐₛₜ (h) | 48.0 | 48.0 | 48.0 | 48.0 | ± | — | — |
| AUC₀₋₁₂ (ng · h/mL) | 417 | 718 | 566 | 567 | ± | 151 | 26.6 |
| AUC₀₋₂₄ (ng · h/mL) | 1429 | 1980 | 1221 | 1543 | ± | 393 | 25.4 |
| AUC₀₋₃₆ (ng · h/mL) | 3035 | 4210 | 1882 | 3042 | ± | 1164 | 38.3 |
| AUC₂₄₋₄₈ (ng · h/mL) | 2644 | 3350 | 1832 | 2609 | ± | 760 | 29.1 |
| AUC₀₋ₗₐₛₜ (ng · h/mL) | 4073 | 5330 | 3053 | 4152 | ± | 1141 | 27.5 |
| AUC_{0-inf} (ng · h/mL) | 4179 | 5450 | 5034 | 4888 | ± | 648 | 13.3 |
| MRT₀₋ₗₐₛₜ (h) | 25.8 | 24.3 | 27.0 | 25.7 | ± | 1.35 | 5.26 |
| MRT_{0-inf} (h) | 26.5 | 24.9 | 46.3 | 32.6 | ± | 11.9 | 36.6 |
| AUC_{Extra} (%) | 2.54 | 2.19 | 39.4 | 14.7 | ± | 21.4 | 145 |
| AUMC_{Extra} (%) | 5.04 | 4.62 | 64.6 | 24.8 | ± | 34.5 | 139 |

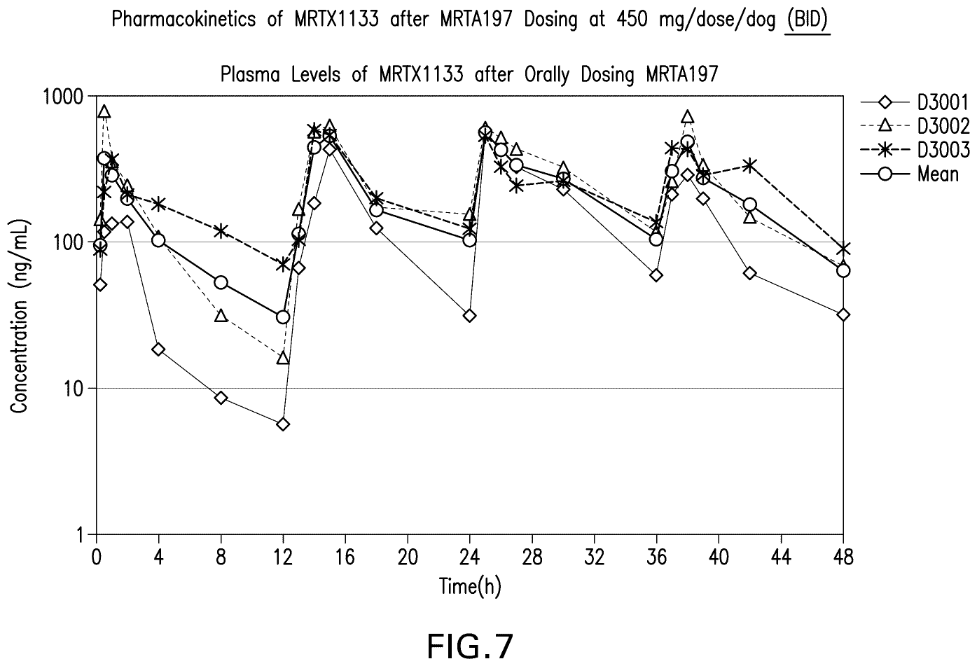

Table 8 lists pharmacokinetic data of MRTA197 formulation. FIG. 7 is a graph of plasma concentration of MRTX1133 after MRTA197 dosing.

TABLE 8
Summary of MRTA197 Pharmacokinetic Parameters
MRTA197

| | D3001 | D3002 | D3003 | Mean | | SD | CV (%) |
|---|---|---|---|---|---|---|---|
| Time (h) | | | | | | | |
| 0 | BQL | BQL | BQL | ND | ± | ND | ND |
| 0.25 | 51.3 | 144 | 90.3 | 95.2 | ± | 46.5 | 48.9 |
| 0.5 | 119 | 787 | 221 | 376 | ± | 360 | 95.8 |
| 1 | 134 | 357 | 370 | 287 | ± | 133 | 46.2 |
| 2 | 138 | 247 | 214 | 200 | ± | 55.9 | 28.0 |
| 4 | 18.5 | 109 | 181 | 103 | ± | 81.4 | 79.2 |
| 8 | 8.58 | 31.4 | 119 | 53.0 | ± | 58.3 | 110 |
| 12 | 5.65 | 16.3 | 69.9 | 30.6 | ± | 34.4 | 112 |
| 13 | 66.9 | 169 | 105 | 114 | ± | 51.6 | 45.4 |
| 14 | 185 | 571 | 581 | 446 | ± | 226 | 50.7 |
| 15 | 431 | 624 | 539 | 531 | ± | 96.7 | 18.2 |
| 18 | 125 | 174 | 199 | 166 | ± | 37.6 | 22.7 |
| 24 | 31.4 | 156 | 123 | 103 | ± | 64.6 | 62.4 |
| 25 | 547 | 602 | 536 | 562 | ± | 35.4 | 6.30 |
| 26 | 436 | 516 | 328 | 427 | ± | 94.3 | 22.1 |
| 27 | 330 | 435 | 244 | 336 | ± | 95.7 | 28.4 |
| 30 | 231 | 324 | 261 | 272 | ± | 47.5 | 17.5 |
| 36 | 59.6 | 117 | 137 | 105 | ± | 40.2 | 38.4 |
| 37 | 213 | 263 | 439 | 305 | ± | 119 | 38.9 |
| 38 | 291 | 733 | 430 | 485 | ± | 226 | 46.6 |
| 39 | 199 | 339 | 286 | 275 | ± | 70.7 | 25.7 |
| 42 | 61.8 | 148 | 334 | 181 | ± | 139 | 76.7 |
| 48 | 31.9 | 68.1 | 90.9 | 63.6 | ± | 29.8 | 46.8 |
| PK Parameters | | | | | | | |
| Rsq_adj | 0.867 | 0.914 | 0.847 | — | ± | — | — |
| No. points used for T_{1/2} | 5.00 | 3.00 | 5.00 | ND | ± | — | — |
| Cₘₐₓ (ng/mL) | 547 | 787 | 581 | 638 | ± | 130 | 20.3 |
| Tₘₐₓ (h) | 25.0 | 0.500 | 14.0 | 13.2 | ± | 12.3 | 93.2 |
| T_{1/2} (h) | 3.43 | 4.05 | 5.04 | 4.17 | ± | 0.812 | 19.5 |
| Tₗₐₛₜ (h) | 48.0 | 48.0 | 48.0 | 48.0 | ± | — | — |
| AUC₀₋₁₂ (ng · h/mL) | 466 | 1384 | 1863 | 1238 | ± | 710 | 57.3 |
| AUC₀₋₂₄ (ng · h/mL) | 2240 | 4423 | 4824 | 3829 | ± | 1391 | 36.3 |
| AUC₀₋₃₆ (ng · h/mL) | 4990 | 8135 | 7724 | 6950 | ± | 1709 | 24.6 |
| AUC₂₄₋₄₈ (ng · h/mL) | 3987 | 6171 | 5995 | 5384 | ± | 1214 | 22.5 |
| AUC₀₋ₗₐₛₜ (ng · h/mL) | 6226 | 10594 | 10820 | 9213 | ± | 2590 | 28.1 |
| AUC_{0-inf} (ng · h/mL) | 6384 | 10991 | 11481 | 9619 | ± | 2812 | 29.2 |
| MRT₀₋ₗₐₛₜ (h) | 25.3 | 24.7 | 25.0 | 25.0 | ± | 0.300 | 1.20 |
| MRT_{0-inf} (h) | 26.0 | 25.7 | 26.7 | 26.1 | ± | 0.513 | 1.96 |
| AUC_{Extra} (%) | 2.48 | 3.62 | 5.76 | 3.95 | ± | 1.67 | 42.1 |
| AUMC_{Extra} (%) | 5.05 | 7.57 | 11.9 | 8.17 | ± | 3.46 | 42.4 |

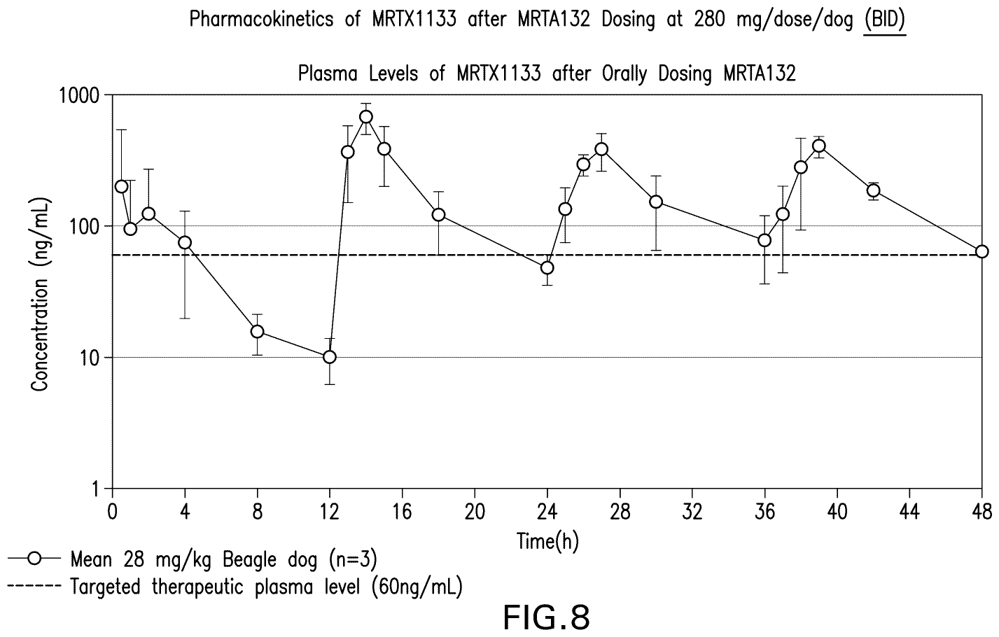

Table 9 lists pharmacokinetic data of MRT-A13-2 formulation. FIG. 8 is a graph of plasma concentration of MRTX1133 after MRT-A13-2 dosing. Capsule dosing was carried out on 3 Beagle dogs (N=3): 70 mg/capsule, 4 capsules/animal, 280 mg/animal/dose, twice a day (BID) for 2 days.

TABLE 9
Summary of MRT-A13-2 Pharmacokinetic Parameters
Plasma concentration (ng/mL)

| | D1001 | D1002 | D1003 | Mean | | SD | CV (%) |
|---|---|---|---|---|---|---|---|
| Time (h) | | | | | | | |
| 0.00 | BQL | BQL | BQL | ND | ± | ND | ND |
| 0.250 | 177 | BQL | BQL | ND | ± | ND | ND |
| 0.500 | 593 | 1.89 | 2.25 | 199 | ± | 341 | 171 |
| 1.00 | 243 | 16.4 | 25.1 | 94.8 | ± | 128 | 135 |
| 2.00 | 62.3 | 290 | 18.9 | 124 | ± | 146 | 118 |
| 4.00 | 39.1 | 138 | 47.0 | 74.7 | ± | 55.0 | 73.6 |
| 8.00 | 11.6 | 21.8 | 13.8 | 15.7 | ± | 5.37 | 34.1 |
| 12.0 | 8.01 | 14.5 | 7.65 | 10.1 | ± | 3.86 | 38.3 |
| 13.0 | 501 | 118 | 474 | 364 | ± | 214 | 58.7 |
| 14.0 | 729 | 476 | 828 | 678 | ± | 182 | 26.8 |
| 15.0 | 260 | 598 | 298 | 385 | ± | 185 | 48.0 |
| 18.0 | 99.7 | 190 | 74.9 | 122 | ± | 60.6 | 49.8 |
| 24.0 | 39.9 | 62.5 | 41.7 | 48.0 | ± | 12.6 | 26.2 |
| 25.0 | 188 | 146 | 69.8 | 135 | ± | 59.9 | 44.5 |
| 26.0 | 261 | 356 | 265 | 294 | ± | 53.7 | 18.3 |
| 27.0 | 248 | 484 | 418 | 383 | ± | 122 | 31.8 |
| 30.0 | 250 | 128 | 80.2 | 153 | ± | 87.6 | 57.3 |
| 36.0 | 121 | 73.7 | 38.3 | 77.7 | ± | 41.5 | 53.4 |
| 37.0 | 203 | 46.3 | 118 | 122 | ± | 78.4 | 64.1 |
| 38.0 | 458 | 291 | 87.5 | 279 | ± | 186 | 66.5 |
| 39.0 | 346 | 490 | 381 | 406 | ± | 75.1 | 18.5 |
| 42.0 | 172 | 217 | 167 | 185 | ± | 27.5 | 14.9 |
| 48.0 | 59.7 | 68.0 | 63.7 | 63.8 | ± | 4.15 | 6.51 |
| PK Parameters | | | | | | | |
| Rsq_adj | 0.989 | 0.984 | 0.958 | — | ± | — | — |
| No. points used for T_{1/2} | 3.00 | 3.00 | 3.00 | 3.00 | ± | — | — |
| Cₘₐₓ (ng/mL) | 729 | 598 | 828 | 718 | ± | 115 | 16.1 |
| Tₘₐₓ (h) | 14.0 | 15.0 | 14.0 | 14.3 | ± | 0.577 | 4.03 |
| T_{1/2} (h) | 3.60 | 3.21 | 3.59 | 3.47 | ± | 0.222 | 6.41 |
| Tₗₐₛₜ (h) | 48.0 | 48.0 | 48.0 | 48.0 | ± | — | — |
| AUC₀₋₁₂ (ng · h/mL) | 722 | 978 | 260 | 653 | ± | 364 | 55.7 |
| AUC₀₋₂₄ (ng · h/mL) | 2940 | 3634 | 2495 | 3023 | ± | 574 | 19.0 |
| AUC₀₋₃₆ (ng · h/mL) | 5326 | 5778 | 3986 | 5030 | ± | 932 | 18.5 |
| AUC₀₋ₗₐₛₜ (ng · h/mL) | 7581 | 8128 | 5779 | 7163 | ± | 1229 | 17.2 |
| AUC_{0-inf} (ng · h/mL) | 7891 | 8443 | 6109 | 7481 | ± | 1220 | 16.3 |
| MRT₀₋ₗₐₛₜ (h) | 26.1 | 25.3 | 26.3 | 25.9 | ± | 0.529 | 2.04 |
| MRT_{0-inf} (h) | 27.2 | 26.3 | 27.8 | 27.1 | ± | 0.755 | 2.79 |
| AUC_{Extra} (%) | 3.93 | 3.73 | 5.39 | 4.35 | ± | 0.906 | 20.8 |
| AUMC_{Extra} (%) | 7.68 | 7.46 | 10.3 | 8.48 | ± | 1.58 | 18.6 |

### Example 3: Pharmacokinetics in Humans

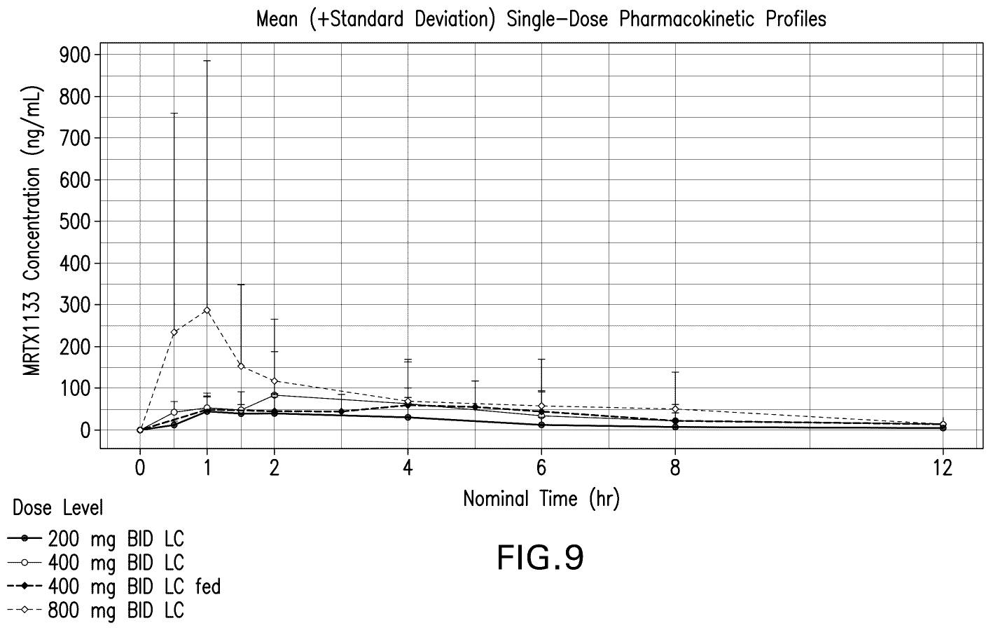

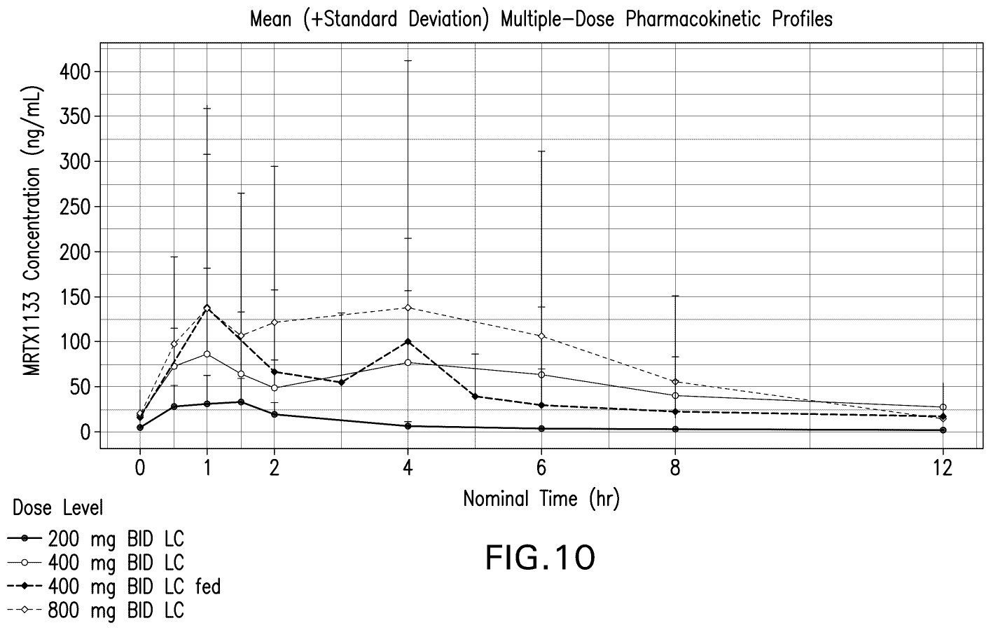

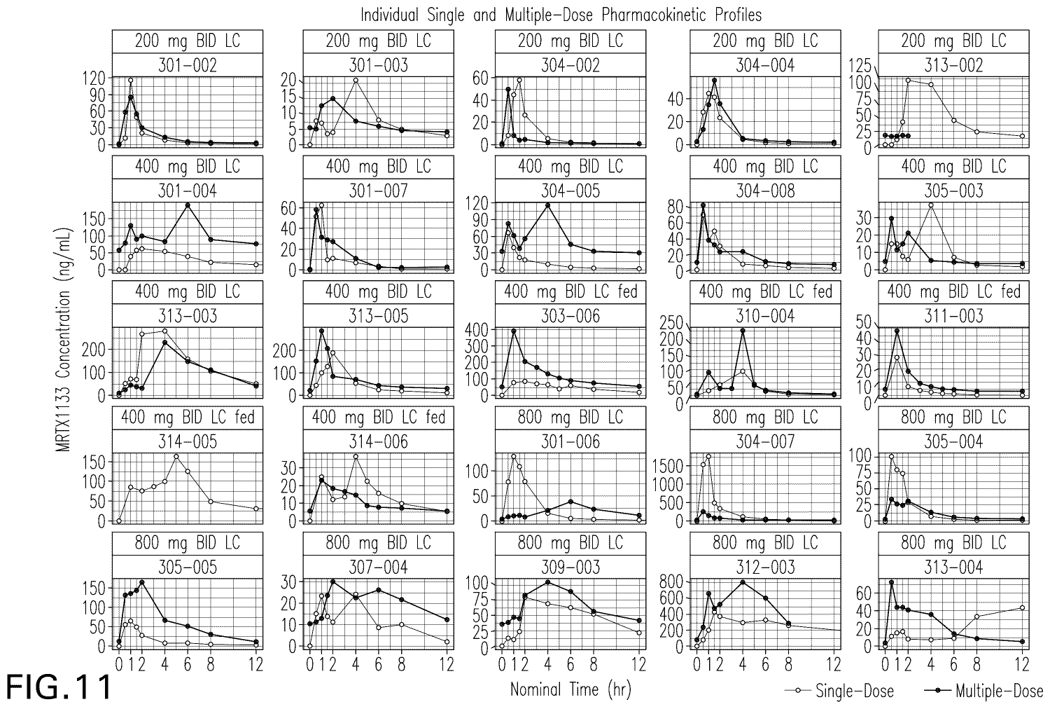

The pharmacokinetics [PK] of the MRTX1133 formulation after oral administration has been evaluated in 25 subjects across 3 escalating dose frequencies under fasting conditions (200 mg twice daily [BID], 400 mg BID, and 800 mg BID), as well as evaluation of 400 mg BID when administered with a low-fat meal (i.e., under fed conditions). The mean (+standard deviation) single-dose and multiple-dose pharmacokinetic profiles of the evaluated doses are presented in FIG. 9 and FIG. 10, respectively. Individual single- and multiple-dose PK profiles can be found in FIG. 11. For all figures, the X-axis represents nominal time of sample collection (hours [h]) and the Y-axis represents plasma concentration (ng/mL). Table 10 lists PK data following the oral administration of a single dose and multiple doses of MRTX1133 formulation by dose level and subject.

When the MRTX1133 dose was increased from 200 mg BID to 400 mg BID under fasted conditions, it resulted in an increase in the mean exposure, approximately 1.8-fold following a single dose and 4.4-fold following multiple doses. However, the mean single- and multiple-dose exposure did not change in a clinically meaningful manner when the dose was increased from 400 mg BID to 800 mg BID under fasted conditions, when accounting for the high degree of inter-patient variability.

The administration of a low-fat meal with 400 mg BID did not result in a clinically meaningful change in exposure compared to administration under fasted conditions.

Overall, the MRTX1133 formulation exhibited very high inter-patient variability across all dose levels, with the % CV ranging from approximately 65 to 200%. A select number of patients receiving MRTX1133 formulation (200 mg BID fasted: 0/5, 400 mg BID fasted: 3/7, 400 mg BID fed: 1/5, 800 mg BID fasted: 3/8) were able to achieve the target human efficacious exposure derived from mouse xenograft models (>40 to 60 ng/mL for ≥8 h of a 12 h dosing interval), following administration of multiple oral doses.

TABLE 10
Summary of MRTX1133 Clinical Pharmacokinetic Parameters
Single oral dose of 200 mg under fasted conditions

| | Subject 1 | Subject 2 | Subject 3 | Subject 4 | Subject 5 | | | | CV |
|---|---|---|---|---|---|---|---|---|---|
| Time (h) | (301-002) | (301-003) | (304-002) | (304-004) | (313-002) | Mean | | SD | (%) |
| 0 | BQL | BQL | BQL | BQL | BQL | NC | ± | NC | NC |
| 0.5 | 12.0 | 7.61 | 8.20 | 27.8 | BQL | 11.1 | ± | 10.3 | 92.5 |
| 1 | 116 | 6.88 | 44.6 | 44.6 | 9.39 | 44.3 | ± | 44.0 | 99.4 |
| 1.5 | 49.8 | 3.38 | 57.9 | 41.4 | 42.7 | 39.0 | ± | 21.0 | 5..8 |
| 2 | 20.4 | 4.01 | 26.7 | 23.5 | 122 | 39.3 | ± | 47.0 | 120 |
| 4 | 7.45 | 21.0 | 4.79 | 4.8 | 114 | 30.4 | ± | 47.2 | 155 |
| 6 | 3.04 | 8.00 | 1.60 | 1.82 | 44.8 | 11.9 | ± | 18.6 | 157 |
| 8 | 2.37 | 4.77 | 0.954 | 1.22 | 23.6 | 6.58 | ± | 9.63 | 146 |
| 12 | 1.73 | 2.86 | 0.678 | 0.912 | 16.0 | 4.44 | ± | 6.52 | 147 |
| 24 | 0.521 | ND | 1.08^{a} | ND | ND | 0.521 | ± | NC | NC |
| 48 | BQL | ND | ND | ND | ND | NC | ± | NC | NC |
| 72 | BQL | ND | ND | ND | ND | NC | ± | NC | NC |
| 96 | BQL | ND | ND | ND | ND | NC | ± | NC | NC |

| | Subject 1 | Subject 2 | Subject 3 | Subject 4 | Subject 5 | Geo | | | Geo CV |
|---|---|---|---|---|---|---|---|---|---|
| PK Parameters | (301-002) | (301-003) | (304-002) | (304-004) | (313-002) | Mean | | SD | (%) |
| Rsq_adj | NC | NC | NC | NC | NC | NC | ± | NC | NC |
| No. points used for T_{1/2} | NC | NC | NC | NC | NC | NC | ± | NC | NC |
| Cₘₐₓ (ng/mL) | 116 | 21.0 | 57.9 | 44.6 | 122 | 59.8 | ± | 44.7 | 83.8 |
| Tₘₐₓ (h)^{b} | 1.0 | 4.0 | 1.5 | 1.0 | 2.0 | 1.5 | ± | 1.0-4.0 | NC |
| T_{1/2} (h) | NC | NC | NC | NC | NC | NC | ± | NC | NC |
| Tₗₐₛₜ (h) | 24 | 12 | 12^{a} | 12 | 12 | NC | ± | NC | NC |
| AUC₀₋₁₂ (ng · h/mL) | 140 | 89.2 | 98.1 | 99.3 | 585 | 148 | ± | 215 | 92.6 |
| AUC₀₋ₗₐₛₜ (ng · h/mL) | 152 | 89.2 | 98.1 | 99.3 | 585 | 151 | ± | 214 | 92.5 |
| AUC_{0-inf}^{c} (ng · h/mL) | 157 | 107 | 103 | 107 | 653 | 165 | ± | 240 | 92.9 |
| AUC_{Extrap} (%) | 3.42 | 16.3 | 4.88 | 7.77 | 10.4 | 7.4 | ± | 5.1 | 67.6 |

Multiple oral doses of 200 mg BID under fasted conditions

| | Subject 1 | Subject 2 | Subject 3 | Subject 4 | Subject 5 | | | | CV |
|---|---|---|---|---|---|---|---|---|---|
| Time (h) | (301-002) | (301-003) | (304-002) | (304-004) | (313-002) | Mean | | SD | (%) |
| 0 | 1.63 | 5.45 | 0.632 | 2.26 | 16.7 | 5.33 | ± | 6.60 | 124 |
| 0.5 | 57.5 | 5.15 | 49.4 | 13.1 | 15.3 | 28.1 | ± | 23.6 | 84.1 |
| 1 | 85.0 | 12.6 | 8.00 | 34.7 | 15.2 | 31.1 | ± | 31.8 | 102 |
| 1.5 | 55.0 | 15.0 | 4.08 | 56.2 | 17.1 | 33.1 | ± | 26.5 | 80.2 |
| 2 | 29.9 | 7.52 | 4.43 | 35.7 | 15.6 | 20.1 | ± | 12.6 | 62.4 |
| 4 | 12.3 | 5.95 | 1.64 | 5.01 | ND | 6.62 | ± | 4.49 | 67.8 |
| 6 | 5.03 | 4.63 | 1.14 | 3.28 | ND | 3.85 | ± | 2.12 | 55.0 |
| 8 | 3.22 | 4.13 | 0.878 | 2.44 | ND | 2.79 | ± | 1.57 | 56.1 |
| 12 | 2.72 | 5.45 | 0.928 | 2.08 | ND | 2.46 | ± | 1.33 | 54.2 |
| 24 | ND | ND | 0.730^{a} | ND | ND | NC | ± | NC | NC |

| | Subject 1 | Subject 2 | Subject 3 | Subject 4 | Subject 5 | Geo | | | Geo CV |
|---|---|---|---|---|---|---|---|---|---|
| PK Parameters | (301-002) | (301-003) | (304-002) | (304-004) | (313-002) | Mean | | SD | (%) |
| Cₘₐₓ (ng/mL) | 85.0 | 15.0 | 49.4 | 56.2 | 17.1 | 36.0 | ± | 29.2 | 89.6 |
| Tₘₐₓ (h)^{b} | 1.0 | 2.0 | 0.5 | 1.5 | 1.5 | 1.5 | ± | 0.5-2.0 | NC |
| Tₗₐₛₜ (h) | 12 | 12 | 12 | 12 | 2.0 | NC | ± | NC | NC |
| AUC₀₋₁₂ (ng · h/mL) | 181 | 84.0 | 42.9 | 115 | NC | 93.1 | ± | 58.4 | 66.6 |
| AUC₀₋ₗₐₛₜ (ng · h/mL) | 181 | 84.0 | 42.9 | 115 | 31.9 | 75.2 | ± | 60.4 | 81.1 |
| Cₘₐₓ Accumulation Ratio | 0.73 | 0.71 | 0.85 | 1.26 | 0.14 | 0.6 | ± | 0.4 | 102 |
| AUC₀₋₁₂ Accumulation Ratio | 1.30 | 0.94 | 0.44 | 1.16 | NC | 0.9 | ± | 0.4 | 52.1 |
| Peak to Trough Ratio | 52.1 | 2.75 | 78.2 | 24.9 | 1.02 | 12.3 | ± | 33.2 | 598 |

Single oral dose of 400 mg under fasted conditions

| | Subject 1 | Subject 2 | Subject 3 | Subject 4 | Subject 5 | Subject 6 | Subject 7 | | | | CV |
|---|---|---|---|---|---|---|---|---|---|---|---|
| Time (h) | (301-004) | (301-007) | (304-005) | (304-008) | (305-003) | (313-003) | (313-005) | Mean | | SD | (%) |
| 0 | BQL | BQL | BQL | BQL | BQL | BQL | BQL | NC | ± | NC | NC |
| 0.5 | BQL | 51.4 | 66.3 | 71 | 15.2 | 51.5 | 43.7 | 42.7 | ± | 26.1 | 61.1 |
| 1 | 39.5 | 62 | 39.8 | 38.2 | 15.2 | 71.7 | 99.1 | 52.2 | ± | 27.6 | 52.8 |
| 1.5 | 57.4 | 9.42 | 22.3 | 49.5 | 7.59 | 69.1 | 127 | 48.9 | ± | 41.9 | 85.8 |
| 2 | 62.1 | 11 | 17.6 | 30.8 | 5.83 | 268 | 188 | 83.3 | ± | 103 | 124 |
| 4 | 54.3 | 6.83 | 9.91 | 7.09 | 37 | 281 | 54.8 | 64.4 | ± | 97.8 | 152 |
| 6 | 39.9 | 2.9 | 4.98 | 5.22 | 6.99 | 158 | 23.8 | 34.5 | ± | 56.1 | 162 |
| 8 | 23 | 0.923 | 3.27 | 3.59 | 2.91 | 106 | 21.2 | 23.0 | ± | 37.7 | 164 |
| 12 | 15.1 | 1.05 | 2.16 | 2.43 | 1.79 | 49.2 | 11.4 | 11.9 | ± | 17.4 | 146 |
| 24 | 3.75 | ND | ND | ND | ND | ND | ND | 3.75 | ± | NC | NC |
| 48 | 1.46 | ND | ND | ND | ND | ND | ND | 1.46 | ± | NC | NC |
| 72 | 0.872 | ND | ND | ND | ND | ND | ND | 0.872 | ± | NC | NC |
| 96 | 0.538 | ND | ND | ND | ND | ND | ND | 0.538 | ± | NC | NC |

| | Subject 1 | Subject 2 | Subject 3 | Subject 4 | Subject 5 | Subject 6 | Subject 7 | Geo | | | Geo CV |
|---|---|---|---|---|---|---|---|---|---|---|---|
| PK Parameters | (301-004) | (301-007) | (304-005) | (304-008) | (305-003) | (313-003) | (313-005) | Mean | | SD | (%) |
| Rsq_adj | 0.999 | NC | NC | NC | NC | NC | NC | NC | ± | NC | NC |
| No. points used for T_{1/2} | 3 | NC | NC | NC | NC | NC | NC | NC | ± | NC | NC |
| Cₘₐₓ (ng/mL) | 62.1 | 62.0 | 66.3 | 71.0 | 37.0 | 281 | 188 | 86.2 | ± | 90.1 | 82.2 |
| Tₘₐₓ (h)^{b} | 2.0 | 1.0 | 0.5 | 0.5 | 4.0 | 4.0 | 2.0 | 2.0 | ± | 0.5-4.0 | NC |
| T_{1/2} (h) | 33.3 | NC | NC | NC | NC | NC | NC | 33.3 | ± | NC | NC |
| Tₗₐₛₜ (h) | 96 | 12 | 12 | 12 | 12 | 12 | 12 | NC | ± | NC | NC |
| AUC₀₋₁₂ (ng · h/mL) | 410 | 94.3 | 128 | 151 | 118 | 1,696 | 580 | 263 | ± | 578 | 146 |
| AUC₀₋ₗₐₛₜ (ng · h/mL) | 610 | 94.3 | 128 | 151 | 118 | 1,696 | 580 | 278 | ± | 580 | 155 |
| AUC_{0-inf}^{c} (ng · h/mL) | 636 | 98.4 | 137 | 169 | 126 | 1,950 | 670 | 304 | ± | 668 | 161 |
| AUC_{Extrap} (%) | 4.07 | 4.18 | 6.90 | 10.7 | 6.70 | 13.0 | 13.4 | 7.6 | ± | 3.9 | 53.3 |

Multiple oral doses of 400 mg BID under fasted conditions

| | Subject 1 | Subject 2 | Subject 3 | Subject 4 | Subject 5 | Subject 6 | Subject 7 | | | | CV |
|---|---|---|---|---|---|---|---|---|---|---|---|
| Time (h) | (301-004) | (301-007) | (304-005) | (304-008) | (305-003) | (313-003) | (313-005) | Mean | | SD | (%) |
| 0 | 57.3 | 0.579 | 33.1 | 9.12 | 4.7 | 9.83 | 21.3 | 19.4 | ± | 20.0 | 103 |
| 0.5 | 79.1 | 58.5 | 82.8 | 83.0 | 29.7 | 23.6 | 152 | 72.7 | ± | 42.8 | 58.9 |
| 1 | 129 | 31.5 | 61.2 | 38.4 | 11.5 | 44.7 | 285 | 85.9 | ± | 95.4 | 111 |
| 1.5 | 90.2 | 28.5 | 38.9 | 32.3 | 15.2 | 38.0 | 209 | 64.6 | ± | 67.9 | 105 |
| 2 | 99.0 | 27.2 | 55.6 | 23.4 | 21.1 | 30.0 | 83.1 | 48.5 | ± | 31.6 | 65.1 |
| 4 | 82.8 | 11.0 | 115 | 23.8 | 5.34 | 233 | 70.5 | 77.3 | ± | 79.8 | 103 |
| 6 | 189 | 2.51 | 45.3 | 10.7 | 4.38 | 148 | 43.8 | 63.4 | ± | 74.8 | 118 |
| 8 | 88.9 | 2.27 | 33.3 | 8.36 | 3.68 | 110 | 35.5 | 40.3 | ± | 43.0 | 107 |
| 12 | 76.9 | 2.76 | 31.2 | 6.42 | 3.71 | 42.1 | 31.3 | 27.8 | ± | 26.8 | 96.4 |
| 24 | ND | ND | ND | ND | ND | ND | ND | NC | ± | NC | NC |

| | Subject 1 | Subject 2 | Subject 3 | Subject 4 | Subject 5 | Subject 6 | Subject 7 | Geo | | | Geo CV |
|---|---|---|---|---|---|---|---|---|---|---|---|
| PK Parameters | (301-004) | (301-007) | (304-005) | (304-008) | (305-003) | (313-003) | (313-005) | Mean | | SD | (%) |
| Cₘₐₓ (ng/mL) | 189 | 58.5 | 115 | 83.0 | 29.7 | 233 | 285 | 111 | ± | 95.5 | 96.6 |
| Tₘₐₓ (h)^{b} | 6.0 | 0.5 | 4.0 | 0.5 | 0.5 | 4.0 | 1.0 | 1.0 | ± | 0.5-6.0 | NC |
| Tₗₐₛₜ (h) | 12 | 12 | 12 | 12 | 12 | 12 | 12 | NC | ± | NC | NC |
| AUC₀₋₁₂ (ng · h/mL) | 1,237 | 128 | 640 | 211 | 89.4 | 1,239 | 821 | 410 | ± | 499 | 153 |
| AUC₀₋ₗₐₛₜ (ng · h/mL) | 1,237 | 128 | 640 | 211 | 89.4 | 1,239 | 821 | 410 | ± | 499 | 153 |
| Cₘₐₓ Accumulation Ratio | 3.04 | 0.94 | 1.73 | 1.17 | 0.80 | 0.83 | 1.52 | 1.3 | ± | 0.8 | 50.8 |
| AUC₀₋₁₂ Accumulation Ratio | 3.02 | 1.35 | 5.02 | 1.40 | 0.76 | 0.73 | 1.41 | 1.6 | ± | 1.5 | 79.4 |
| Peak to Trough Ratio | 3.30 | 101 | 3.47 | 9.10 | 6.32 | 23.7 | 13.4 | 11.1 | ± | 35.2 | 180 |

Single oral dose of 400 mg with a low-fat meal

| | Subject 1 | Subject 2 | Subject 3 | Subject 4 | Subject 5 | | | | CV |
|---|---|---|---|---|---|---|---|---|---|
| Time (h) | (303-006) | (310-004) | (311-003) | (314-005) | (314-006) | Mean | | SD | (%) |
| 0 | BQL | BQL | BQL | BQL | BQL | NC | ± | NC | NC |
| 1 | 77.0 | 18.7 | 28.2 | 85.1 | 24.7 | 46.7 | ± | 31.6 | 67.7 |
| 2 | 83.2 | 39.9 | 6.47 | 76.4 | 12.0 | 43.6 | ± | 35.5 | 81.4 |
| 3 | 71.5 | ND | 3.84 | 85.8 | 13.6 | 43.7 | ± | 41.0 | 93.8 |
| 4 | 65.9 | 93.9 | 2.43 | 99.8 | 36.5 | 59.7 | ± | 40.7 | 68.2 |
| 5 | 41.4 | 41.4 | 1.41 | 163 | 22.5 | 53.9 | ± | 63.2 | 117 |
| 6 | 59.5 | 16.9 | 1.02 | 125 | 15.5 | 43.6 | ± | 50.5 | 116 |
| 8 | 36.2 | 6.62 | 0.538 | 48.8 | 9.72 | 20.4 | ± | 20.9 | 103 |
| 12 | 17.5 | 3.17 | BQL | 30.4 | 5.32 | 11.3 | ± | 12.6 | 112 |
| 24 | ND | ND | ND | 7.59 | ND | 7.59 | ± | NC | NC |
| 48 | ND | ND | ND | 3.13 | ND | 3.13 | ± | NC | NC |
| 72 | ND | ND | ND | 2.13 | ND | 2.13 | ± | NC | NC |
| 96 | ND | ND | ND | 1.12 | ND | 1.12 | ± | NC | NC |

| | Subject 1 | Subject 2 | Subject 3 | Subject 4 | Subject 5 | Geo | | | Geo CV |
|---|---|---|---|---|---|---|---|---|---|
| PK Parameters | (303-006) | (310-004 | (311-003) | (314-005) | (314-006) | Mean | | SD | (%) |
| Rsq_adj | NC | NC | NC | 0.966 | NC | NC | ± | NC | NC |
| No. points used for T_{1/2} | NC | NC | NC | 4 | NC | NC | ± | NC | NC |
| Cₘₐₓ (ng/mL) | 83.2 | 93.9 | 28.2 | 163 | 36.5 | 66.6 | ± | 54.0 | 82.3 |
| Tₘₐₓ (h)^{b} | 2.0 | 4.0 | 1.0 | 5.0 | 4.0 | 4.0 | ± | 1.0 | 5.0 |
| T_{1/2} (h) | NC | NC | NC | 27.2 | NC | 27.2 | ± | NC | NC |
| Tₗₐₛₜ (h) | 12 | 12 | 8 | 96 | 12 | NC | ± | NC | NC |
| AUC₀₋₁₂ (ng · h/mL) | 564 | 305 | 42.6 | 889 | 169 | 256 | ± | 338 | 174 |
| AUC₀₋ₗₐₛₜ (ng · h/mL) | 564 | 305 | 42.6 | 1,307 | 169 | 277 | ± | 503 | 207 |
| AUC_{0-inf}^{c} (ng · h/mL) | 651 | 317 | 43.2 | 1,351 | 200 | 300 | ± | 519 | 211 |
| AUC_{Extrap} (%) | 13.4 | 3.77 | 3.88 | 3.25 | 15.3 | 6.3 | ± | 5.9 | 87.8 |

Multiple oral doses of 400 mg BID with a low-fat meal

| | Subject 1 | Subject 2 | Subject 3 | Subject 4 | Subject 5 | | | | CV |
|---|---|---|---|---|---|---|---|---|---|
| Time (h) | (303-006) | (310-004) | (311-003) | (314-005) | (314-006) | Mean | | SD | (%) |
| 0 | 53.8 | 2.95 | 4.53 | ND | 5.39 | 16.7 | ± | 24.8 | 149 |
| 1 | 390 | 90.3 | 48.2 | ND | 23.0 | 138 | ± | 170 | 124 |
| 2 | 203 | 27.3 | 17.9 | ND | 18.5 | 66.7 | ± | 91.0 | 136 |
| 3 | 170 | 24.8 | 9.11 | ND | 16.6 | 55.1 | ± | 76.8 | 139 |
| 4 | 129 | 250 | 6.60 | ND | 14.7 | 100 | ± | 115 | 114 |
| 5 | 106 | 39.4 | 5.09 | ND | 8.57 | 39.8 | ± | 46.8 | 118 |
| 6 | 89.4 | 18.8 | 4.07 | ND | 7.64 | 30.0 | ± | 40.1 | 134 |
| 8 | 73.2 | 8.47 | 3.12 | NI | 7.05 | 23.0 | ± | 33.6 | 146 |
| 12 | 55.3 | 5.28 | 2.90 | ND | 5.71 | 17.3 | ± | 25.4 | 147 |
| 24 | ND | ND | ND | ND | ND | NC | ± | NC | NC |

| | Subject 1 | Subject 2 | Subject 3 | Subject 4 | Subject 5 | Geo | | | Geo CV |
|---|---|---|---|---|---|---|---|---|---|
| PK Parameters | (303-006) | (310-004) | (311-003) | (314-005) | (314-006) | Mean | | SD | (%) |
| Cₘₐₓ (ng/mL) | 390 | 250 | 48.2 | NC | 23.0 | 102 | ± | 174 | 224 |
| Tₘₐₓ (h)^{b} | 1.0 | 4.0 | 1.0 | NC | 1.0 | 1.0 | ± | 1.0-4.0 | NC |
| Tₗₐₛₜ (h) | 12 | 12 | 12 | NC | 12 | NC | ± | NC | NC |
| AUC₀₋₁₂ (ng · h/mL) | 1,475 | 457 | 107 | NC | 127 | 310 | ± | 642 | 187 |
| AUC₀₋ₗₐₛₜ (ng · h/mL) | 1,475 | 457 | 107 | NC | 127 | 310 | ± | 642 | 187 |
| Cₘₐₓ Accumulation Ratio | 4.69 | 2.66 | 1.71 | NC | 0.63 | 1.9 | ± | 1.7 | 103 |
| AUC₀₋₁₂ Accumulation Ratio | 2.61 | 1.50 | 2.52 | NC | 0.75 | 1.7 | ± | 0.9 | 63.4 |
| Peak to Trough Ratio | 7.25 | 84.7 | 10.6 | NC | 4.27 | 12.9 | ± | 38.8 | 213 |

Single oral dose of 800 mg under fasted conditions

| | Subject 1 | Subject 2 | Subject 3 | Subject 4 | Subject 5 | Subject 6 | Subject 7 | Subject 8 | | | | CV |
|---|---|---|---|---|---|---|---|---|---|---|---|---|
| Time (h) | (301-006) | (304-007) | (305-004) | (305-005) | (307-004) | (309-003) | (312-003) | (313-004) | Mean | | SD | (%) |
| 0 | BQL | BQL | 0 | BQL | BQL | BQL | BQL | BQL | NC | ± | NC | NC |
| 0.5 | 77.9 | 1530 | 101 | 55.3 | 14.9 | 13.1 | 78.5 | 11.2 | 235 | ± | 524 | 223 |
| 1 | 129 | 1760 | 79.9 | 64.3 | 23.4 | 11.4 | 205 | 15.4 | 286 | ± | 599 | 209 |
| 1.5 | 108 | 489 | 74.2 | 48.9 | 13.8 | 24 | 437 | 16.2 | 151 | ± | 195 | 129 |
| 2 | 78.2 | 328 | 29.3 | 27.7 | 11.2 | 80.5 | 374 | 8.36 | 117 | ± | 147 | 126 |
| 4 | 14.7 | 108 | 6.77 | 8.95 | 24 | 69.2 | 302 | 6.89 | 67.6 | ± | 101 | 150 |
| 6 | 4.95 | 37.3 | 3.2 | 8.44 | 8.62 | 62.5 | 329 | 8.79 | 57.9 | ± | 111 | 193 |
| 8 | 2.73 | 26 | 1.12 | 4.28 | 10.1 | 53.2 | 266 | 33.8 | 49.7 | ± | 89.3 | 180 |
| 12 | 2.06 | 14.4 | 1.22 | 2.51 | 2.1 | 22.3 | ND | 43.3 | 12.6 | ± | 15.8 | 126 |
| 24 | 0.894 | ND | ND | ND | ND | ND | 50.2 | ND | 25.5 | ± | 34.9 | 136 |
| 48 | BQL | ND | ND | ND | ND | ND | ND | ND | NC | ± | NC | NC |
| 72 | BQL | ND | ND | ND | ND | ND | ND | ND | NC | ± | NC | NC |
| 96 | BQL | ND | ND | ND | ND | ND | ND | ND | NC | ± | NC | NC |

| | Subject 1 | Subject 2 | Subject 3 | Subject 4 | Subject 5 | Subject 6 | Subject 7 | Subject 8 | Geo | | | Geo CV |
|---|---|---|---|---|---|---|---|---|---|---|---|---|
| PK Parameters | (301-006) | (304-007) | (305-004) | (305-005) | (307-004) | (309-003) | (312-003) | (313-004) | Mean | | SD | (%) |
| Rsq_adj | NC | NC | NC | NC | NC | NC | NC | NC | NC | ± | NC | NC |
| No. points | NC | NC | NC | NC | NC | NC | NC | NC | NC | ± | NC | NC |
| used for T_{1/2} | | | | | | | | | | | | |
| Cₘₐₓ (ng/mL) | 129 | 1,760 | 101 | 64.3 | 24.0 | 80.5 | 437 | 43.3 | 123 | ± | 593 | 234 |
| Tₘₐₓ (h)^{b} | 1.0 | 1.0 | 0.5 | 1.0 | 4.0 | 2.0 | 1.5 | 12 | 1.2 | ± | 0.5-12 | NC |
| T_{1/2} (h) | NC | NC | NC | NC | NC | NC | NC | NC | NC | ± | NC | NC |
| Tₗₐₛₜ (h) | 24 | 12 | 12 | 12 | 12 | 12 | 24 | 12 | NC | ± | NC | NC |
| AUC₀₋₁₂ | 287 | 2,573 | 182 | 167 | 133 | 583 | 3,221 | 251 | 440 | ± | 1,237 | 192 |
| (ng · h/mL) | | | | | | | | | | | | |
| AUC₀₋ₗₐₛₜ | 315 | 2,573 | 182 | 167 | 133 | 583 | 4,421 | 251 | 463 | ± | 1,579 | 215 |
| (ng · h/mL) | | | | | | | | | | | | |
| AUC_{0-inf}^{c} | 317 | 2,665 | 185 | 181 | 141 | 708 | 4,902 | NC | 543 | ± | 1,826 | 251 |
| (ng · h/mL) | | | | | | | | | | | | |
| AUC_{Extrap} (%) | 4.05 | 3.44 | 1.58 | 8.08 | 5.77 | 17.7 | 9.81 | NC | 5.6 | ± | 5.4 | 92.6 |

Multiple oral doses of 800 mg BID under fasted conditions

| | Subject 1 | Subject 2 | Subject 3 | Subject 4 | Subject 5 | Subject 6 | Subject 7 | Subject 8 | | | | CV |
|---|---|---|---|---|---|---|---|---|---|---|---|---|
| Time (h) | (301-006) | (304-007) | (305-004) | (305-005) | (307-004) | (309-003) | (312-003) | (313-004) | Mean | | SD | (%) |
| 0 | 2.55 | 19.3 | 2.01 | 12 | 10.6 | 36.5 | 78.1 | 3.63 | 20.6 | ± | 25.9 | 126 |
| 0.5 | 7.77 | 241 | 34.4 | 131 | 11.2 | 38.8 | 241 | 72.6 | 97.2 | ± | 97.0 | 99.8 |
| 1 | 9.9 | 149 | 25.9 | 136 | 12.9 | 47.4 | 670 | 44.1 | 137 | ± | 222 | 162 |
| 1.5 | 10.7 | 80.9 | 24.5 | 143 | 23.5 | 45.1 | 483 | 44.3 | 107 | ± | 158 | 148 |
| 2 | 8.1 | 84.2 | 30.8 | 165 | 29.8 | 83.3 | 531 | 41 | 122 | ± | 173 | 142 |
| 4 | 19.9 | 26.3 | 13.4 | 67.4 | 22.5 | 105 | 812 | 35.9 | 138 | ± | 274 | 199 |
| 6 | 38.2 | 18.9 | 4.94 | 51.2 | 26.1 | 89.7 | 609 | 14.1 | 107 | ± | 205 | 192 |
| 8 | 22.7 | 18.1 | 3.5 | 30.1 | 21.6 | 57 | 288 | 8.66 | 56.2 | ± | 95.0 | 169 |
| 12 | 11.1 | 16.8 | 2.59 | 12.1 | 12.4 | 42.7 | ND | 5.14 | 14.7 | ± | 13.2 | 90.1 |
| 24 | ND | ND | ND | ND | ND | ND | ND | ND | NC | ± | NC | NC |

| | Subject 1 | Subject 2 | Subject 3 | Subject 4 | Subject 5 | Subject 6 | Subject 7 | Subject 8 | Geo | | | Geo CV |
|---|---|---|---|---|---|---|---|---|---|---|---|---|
| PK Parameters | (301-006) | (304-007) | (305-004) | (305-005) | (307-004) | (309-003) | (312-003) | (313-004) | Mean | | SD | (%) |
| Cₘₐₓ (ng/mL) | 38.2 | 241 | 34.4 | 165 | 29.8 | 105 | 812 | 72.6 | 99.5 | ± | 263 | 163 |
| Tₘₐₓ (h)^{b} | 6.0 | 0.5 | 0.5 | 2.0 | 2.0 | 4.0 | 4.0 | 0.5 | 2.0 | ± | 0.5-6.0 | NC |
| Tₗₐₛₜ (h) | 12 | 12 | 12 | 12 | 12 | 12 | 8 | 12 | NC | ± | NC | NC |
| AUC₀₋₁₂ | 227 | 509 | 130 | 744 | 248 | 820 | NC | 264 | 347 | ± | 273 | 77.2 |
| (ng · h/mL) | | | | | | | | | | | | |
| AUC₀₋ₗₐₛₜ | 227 | 509 | 130 | 744 | 248 | 820 | 4,458 | 264 | 477 | ± | 1,450 | 130 |
| (ng · h/mL) | | | | | | | | | | | | |
| Cₘₐₓ | 0.30 | 0.14 | 0.34 | 2.57 | 1.24 | 1.30 | 1.86 | 1.68 | 0.8 | ± | 0.9 | 144 |
| Accumulation | | | | | | | | | | | | |
| Ratio | | | | | | | | | | | | |
| AUC₀₋₁₂ | 0.79 | 0.20 | 0.71 | 4.46 | 1.87 | 1.41 | NC | 1.05 | 1.0 | ± | 0.9 | 123 |
| Accumulation | | | | | | | | | | | | |
| Ratio | | | | | | | | | | | | |
| Peak to | 15.0 | 12.5 | 17.1 | 13.8 | 2.81 | 2.88 | 10.4 | 20.0 | 9.6 | ± | 6.2 | 91.2 |
| Trough Ratio | | | | | | | | | | | | |

^{a}Sample taken after PM dose. Concentration excluded from analysis and summary statistics.
^{b}Median and Min-Max presented rather than Mean and SD.
^{c}AUC_{inf} not reported and excluded from summary statistics if % AUC extrapolated >20%.
^{d}Excluded from summary statistics due to incomplete PK sampling.

All publications and patent applications cited in this specification are herein incorporated by reference as if each individual publication or patent application were specifically and individually indicated to be incorporated by reference. Although the foregoing has been described in some detail by way of illustration and example for purposes of clarity of understanding, it will be readily apparent to those of ordinary skill in the art in light of the teachings of the specification that certain changes and modifications may be made thereto without departing from the spirit or scope of the appended claims.

1. A solid pharmaceutical composition, suitable for oral administration to a subject, which comprises MRTX1133 ((4-(4-((1R,5S)-3,8-diazabicyclo[3.2.1]octan-3-yl)-8-fluoro-2-(((2R,7aS)-2-fluorohexahydro-1Hpyrrolizin-7a-yl)methoxy)pyrido[4,3-d]pyrimidin-7-yl)-5-ethynyl-6-fluoronaphthalen-2-ol) or a pharmaceutically acceptable salt thereof, wherein the solid pharmaceutical composition, after administration to the subject, is capable of providing AUC_{0→∞} (the area under the curve of a plot of plasma drug concentration versus time) for MRTX1133 of at least, or about, 6900 ng*hr/mL.

2.-6. (canceled)

7. A solid pharmaceutical composition, suitable for oral administration to a subject, which comprises MRTX1133 or a pharmaceutically acceptable salt thereof, wherein the solid pharmaceutical composition, after administration to the subject, is capable of providing Cₘₐₓ for MRTX1133 of at least, or about, 710 ng/mL.

8. A solid pharmaceutical composition, suitable for oral administration to a subject, which comprises MRTX1133 or a pharmaceutically acceptable salt thereof, wherein the solid pharmaceutical composition, after administration to the subject, is capable of providing: a) AUC_{0→∞} for MRTX1133 of at least, or about, 6900 ng*hr/mL; b) AUC_{0→last} for MRTX1133 of at least, or about, 6700 ng*hr/mL; c) AUC_{24→48} for MRTX1133 of at least, or about, 3900 ng*hr/mL; d) AUC_{0→36} for MRTX1133 of at least, or about, 4700 ng*hr/mL; e) AUC_{0→24} for MRTX1133 of at least, or about, 2800 ng*hr/mL; f) AUC_{0→12} for MRTX1133 of at least, or about, 700 ng*hr/mL; and g) Cₘₐₓ for MRTX1133 of at least, or about, 710 ng/mL.

9. The solid pharmaceutical composition of any one of claim 1 wherein the solid pharmaceutical composition is in the form of a capsule.

10. The solid pharmaceutical composition of claim 9, wherein MRTX1133 constitutes 5-50% by the weight of the composition.

11.-13. (canceled)

14. The solid pharmaceutical composition of claim 9, wherein the pharmaceutical composition comprises:
(1) MRTX1133 constituting about 10-40% of the composition;
(2) sucrose acetate isobutyrate (SAIB) constituting about 35-60% of the composition;
(3) an antioxidant constituting about 0-5% of the composition;
(4) a solvent constituting about 10% of the composition;
(5) an emulsifier constituting about 0-15% of the composition; and
(6) a surfactant constituting up to about 10-25% of the composition;
wherein all percentages are percentages by weight and wherein the total weight is 100%.

15.-21. (canceled)

22. The solid pharmaceutical composition of any one of claim 1, wherein the amount of MRTX1133 in the solid pharmaceutical composition is at least, or about, 25 mg.

23. The solid pharmaceutical composition of any one of claim 1, wherein the amount of MRTX1133 in the solid pharmaceutical composition is at least, or about, 50 mg.

24. The solid pharmaceutical composition of any one of claim 1, wherein the amount of MRTX1133 in the solid pharmaceutical composition is at least, or about, 100 mg.

25. The solid pharmaceutical composition of any one of claim 1, wherein the amount of MRTX1133 in the solid pharmaceutical composition is at least, or about, 150 mg.

26. The solid pharmaceutical composition of any one of claim 1, wherein the amount of MRTX1133 in the solid pharmaceutical composition is at least, or about, 200 mg.

27. The solid pharmaceutical composition of any one of claim 1, wherein the amount of MRTX1133 in the solid pharmaceutical composition is at least, or about, 300 mg.

28. The solid pharmaceutical composition of any one of claim 1, wherein the amount of MRTX1133 in the solid pharmaceutical composition is at least, or about, 400 mg.

29. The solid pharmaceutical composition of any one of claim 1, wherein the amount of MRTX1133 in the solid pharmaceutical composition is at least, or about, 600 mg.

30. The solid pharmaceutical composition of claim 14, comprising
(1) MRTX1133 at about 20% of the composition;
(2) SAIB at about 35% of the composition;
(3) propyl gallate at about 5% of the composition;
(4) ethanol at about 10% of the composition;
(5) Lauroyl Polyoxyl-32 glycerides at about 5% of the composition;
(6) Vitamin E TPGS at about 10% of the composition; and
(7) Caprylocaproyl Polyoxyl-8 glycerides at about 15% of the composition;
wherein all percentages are percentages by weight and wherein the total weight is 100%.

31. The solid pharmaceutical composition of claim 14, comprising
(1) MRTX1133 at about 20% of the composition;
(2) SAIB at about 35% of the composition;
(3) propyl gallate at about 5% of the composition;
(4) ethanol at about 10% of the composition;
(5) Lauroyl Polyoxyl-32 glycerides at about 10% of the composition; and
(6) Vitamin E TPGS at about 20% of the composition;
wherein all percentages are percentages by weight and wherein the total weight is 100%.

32. The solid pharmaceutical composition of claim 14, comprising
(1) MRTX1133 at about 20% of the composition;
(2) SAIB at about 35% of the composition;
(3) propyl gallate at about 5% of the composition;
(4) ethanol at about 10% of the composition;
(5) Caprylocaproyl Polyoxyl-8 glycerides at about 15% of the composition; and
(6) Vitamin E TPGS at about 15% of the composition;
wherein all percentages are percentages by weight and wherein the total weight is 100%.

33. The solid pharmaceutical composition of claim 14, comprising
(1) MRTX1133 at about 20% of the composition;
(2) SAIB at about 35% of the composition;
(3) ethanol at about 10% of the composition;
(4) Lauroyl Polyoxyl-32 glycerides at about 5% of the composition
(5) Vitamin E TPGS at about 15% of the composition;
(6) Caprylocaproyl Polyoxyl-8 glycerides at about 15% of the composition; and
wherein all percentages are percentages by weight and wherein the total weight is 100%.

34. The solid pharmaceutical composition of claim 14, comprising
(1) MRTX1133 at about 20% of the composition;
(2) SAIB at about 40% of the composition;
(3) ethanol at about 10% of the composition;
(4) Lauroyl Polyoxyl-32 glycerides at about 5% of the composition
(5) Caprylocaproyl Polyoxyl-8 glycerides at about 20% of the composition; and
wherein all percentages are percentages by weight and wherein the total weight is 100%.

35. The solid pharmaceutical composition of claim 14, wherein the solvent comprises ethanol.

36. The solid pharmaceutical composition of claim 14, wherein the antioxidant comprises propyl gallate.

37. The solid pharmaceutical composition of claim 14, wherein the emulsifier comprises Vitamin E TPGS.

38. The solid pharmaceutical composition of claim 14, wherein the surfactant comprises Lauroyl Polyoxyl-32 glycerides and Caprylocaproyl Polyoxyl-8 glycerides.

39. A solid capsule pharmaceutical composition comprising MRTX1133 prepared by a process which comprises the steps of:
(a) mixing MRTX1133 and one or more solvents together to dissolve MRTX1133;
(b) adding an antioxidant, a surfactant and an emulsifier to the mixture of step (a) and stirring the mixture until all ingredients are dissolved;
(c) adding SAIB and a solvent to the mixture of step (b) and stirring until a uniform solution is formed;
(d) removing volatile solvents from the mixture of step (c); and
(e) encapsulating the solution of step (d) in capsules.

40. The solid capsule pharmaceutical composition of claim 39, wherein the removal of volatile solvents in step (d) comprises reduced pressure evaporation.

41. A method of treating cancer in a subject in need thereof, the method comprising administering to the subject a therapeutically effective amount of the solid pharmaceutical composition of any one of claim 1.

42. The method of claim 41, wherein the therapeutically effective amount is at least, or about, 450 mg of MRTX1133.

43. (canceled)

44. The method of claim 41, wherein the cancer is lung cancer.

45. A method for treating cancer in a subject in need thereof, the method comprising (a) determining that cancer is associated with a KRas G12D mutation; and (b) administering to the subject a therapeutically effective amount of the solid pharmaceutical composition of any one of claim 1.

46. A pharmaceutical composition comprising:
(1) MRTX1133 or a pharmaceutically acceptable salt thereof, and,
(2) sucrose acetate isobutyrate (SAIB).

47. The pharmaceutical composition of claim 46, further comprising an antioxidant.

48. (canceled)

49. The pharmaceutical composition of claim 46 further comprising one or more emulsifiers.

50. The pharmaceutical composition of claim 46 further comprising a surfactant.

51. (canceled)

52. The pharmaceutical composition of claim 47 wherein said sucrose acetate isobutyrate (SAIB) constitutes about 15-60% of total weight of said pharmaceutical composition.

53. The pharmaceutical composition of claim 52, wherein said antioxidant constitutes about 0-10% of total weight of said pharmaceutical composition.

54-56. (canceled)

57. The pharmaceutical composition according to claim 47 wherein said antioxidant is propyl gallate.

58. (canceled)

59. The pharmaceutical composition according to claim 49 wherein said emulsifier is labrasol.

60. The pharmaceutical composition according to claim 50 wherein said surfactant is Gelucire 44/14.
